(19) United States
(12) Patent Application Publication
(10) Pub. No.: US 2020/0267936 A1
(43) Pub. Date: Aug. 27, 2020

(54) PET MONITORING

(71) Applicant: Bao Tran, San Jose, CA (US)

(72) Inventor: Bao Tran, San Jose, CA (US)

(21) Appl. No.: 16/286,539

(22) Filed: Feb. 26, 2019

(51) Int. Cl. A01K 15/02; A01K 29/00; A01K 11/00; A01K 27/00

(52) U.S. Cl. CPC A01K 15/029; A01K 29/005; A01K 11/004; A61B 5/0006; A01K 27/001; A01K 15/021; A01K 11/008

(57) ABSTRACT

A system to monitor a pet includes a wearable device with: sensor(s) to monitor vital sign; a wireless transceiver to determine a geolocation of the pet; a processor coupled to the sensor and the wireless transceiver, the processor capturing pet parameters for the pet's caregiver or owner.

### FIELD OF THE INVENTION

The present invention relates generally to monitoring of pets.

### BACKGROUND OF THE INVENTION

Pets are part of our everyday lives and part of our families. They provide us with companionship but also with emotional support, reduce our stress levels, sense of loneliness and help us to increase our social activities and add to a child's self-esteem and positive emotional development. However, with great benefits come great responsibility, among which includes health monitoring, tracking, or potty training of pets such as cats and dogs.

### SUMMARY OF THE INVENTION

In one aspect, a system to monitor a pet includes a collar including: sensor(s) to monitor vital sign; a wireless transceiver to determine a geolocation of the pet; a processor coupled to the sensor and the wireless transceiver, the processor capturing pet parameters for the pet's caregiver or owner.

In another aspect, a system to monitor a pet includes a collar including: sensor(s) to monitor vital signs including heart rate and motion; a feedback module adapted to transmit feedback to the pet; a wireless transceiver to determine a geolocation of the pet; a processor coupled to the sensor and the wireless transceiver, the processor capturing pet parameters for the pet's caregiver or owner.

In another aspect, a system to train a pet includes a collar including: an odor sensor; an electric shock module adapted to transmit an electrical discharge to the pet; a wireless transceiver to determine a geolocation of the pet; a processor coupled to the odor sensor, the electric shock generator, and the wireless transceiver, the processor detecting if the pet emits a predetermined odor within a preselected area and activating the electric shock generator in response thereto; and a geo-fencing module wirelessly coupled to the processor through the wireless transceiver to define boundary of the predetermined area.

In another aspect, a system to train a pet has a collar including: an odor sensor; a sound generator module adapted to transmit an annoying noise to the pet, wherein said annoying noise is outside of human hearable frequency; a wireless transceiver to determine a geolocation of the pet; a processor coupled to the odor sensor, the sound generator, and the wireless transceiver, the processor detecting if the pet emits a predetermined odor within a preselected area and activating the sound generator in response thereto as a negative feedback to the pet; and a geo-fencing module wirelessly coupled to the processor through the wireless transceiver to define boundary of the predetermined area.

In a further aspect, a pet collar system and method for the remote control and training of a pet or other suitable animal to pee or to poop only in a selected geographical boundary. The system uses a series of audible cues or electrical shocks to motivate the pet to move away from an approaching preselected boundary while continually monitoring the current indoor/outdoor location of the pet and recording those positions.

While the above aspects are in a wearable housing such as a collar, a chest strap, a foot strap, a pet smart clothing, the system can also be in-the-ear devices that captures pet metrics including heart rate, breathing rate, and activity. The devices offer an easy and more natural way of regularly capturing precise data in daily activities.

In another aspect, systems and methods for assisting a pet include a housing custom fitted to a pet anatomy; a microphone to capture sound coupled to a processor to deliver enhanced sound to the pet anatomy; an amplifier with gain and amplitude controls for each hearing frequency; and a learning machine (such as a neural network) to identify an aural environment (such as a park, a car, or home environment) and adjusting amplifier controls to optimize hearing based on the identified aural environment. In one embodiment, the environment can be identified by the background noise or inferred through GPS location, for example.

In another aspect, a method for assisting a pet includes customizing an in-ear device to a pet anatomy; capturing sound using the in-ear device; enhancing sound based on predetermined profiles and transmitting the sound to an ear drum.

In yet another aspect, a method for assisting a pet includes customizing an in-ear device to a pet anatomy; capturing sound using the in-ear device; capturing vital signs with the in-ear device; and learning health signals from the sound and the vital signs from the in-ear device.

In a further aspect, a method includes customizing an in-ear device to a pet anatomy; capturing vital signs with the in-ear device; and learning health signals from the vital signs from the in-ear device.

In another aspect, a method includes customizing an in-ear device to a pet anatomy; capturing vital signs to detect biomarkers with the in-ear device; correlating genomic disease markers with the detected biomarkers to predict health with the in-ear device.

In another aspect, a method includes customizing an in-ear device to a pet anatomy; identifying genomic disease markers; capturing vital signs to detect biomarkers with the in-ear device; correlating genomic disease markers with the detected biomarkers to predict health with the in-ear device.

In another aspect, a method includes customizing an in-ear device to a pet anatomy; capturing accelerometer data and vital signs; controlling a virtual reality device or augmented reality device with acceleration or vital sign data from the in-ear device.

In another aspect, a method includes customizing an in-ear device to a pet anatomy; capturing heart rate, EEG or ECG signal with the in-ear device; and determining pet intent with the in-ear device. The determined pet intent can be used to control an appliance, or can be used to indicate interest for advertisers.

In another aspect, a method includes customizing an in-ear device to a pet anatomy; capturing heart rate, EEG/ECG signal or temperature data to detect biomarkers with the in-ear device; and predict health with the in-ear device data.

In another aspect, a method includes customizing an in-ear device to a pet anatomy; capturing sounds from an advertisement, capturing vital signs associated with the advertisement; and customizing the advertisement to attract the user.

In another aspect, a method includes customizing an in-ear device to a pet anatomy; capturing vital signs associated with a situation; detecting pet emotion from the vital signs; and customizing an action based on pet emotion. In one embodiment, such detected pet emotion is provided to a robot to be more responsive to the user.

In another aspect, a method includes customizing an in-ear device to a pet anatomy; capturing a command from a user, detecting pet emotion based on vital signs; and performing an action in response to the command and the detected pet emotion.

In another aspect, a method includes customizing an in-ear device to a pet anatomy; capturing a command from a user, authenticating the user based on a voiceprint or pet vital signs; and performing an action for the pet in response to the command.

In one aspect, a method for assisting a pet includes customizing an in-ear device to a pet anatomy; capturing sound using the in-ear device; enhancing sound based on predetermined profiles and transmitting the sound to an ear drum.

In one aspect, a method for assisting a pet includes providing an in-ear device to a pet anatomy; capturing sound using the in-ear device; capturing vital signs with the in-ear device; and learning health signals from the sound and the vital signs from the in-ear device.

In another aspect, a method includes providing an in-ear device to a pet anatomy; capturing vital signs with the in-ear device; and learning health signals from the vital signs from the in-ear device.

In another aspect, a method includes providing an in-ear device to a pet anatomy; capturing vital signs to detect biomarkers with the in-ear device; correlating genomic disease markers with the detected biomarkers to predict health with the in-ear device.

In another aspect, a method includes providing an in-ear device to a pet anatomy; identifying genomic disease markers; capturing vital signs to detect biomarkers with the in-ear device; correlating genomic disease markers with the detected biomarkers to predict health with the in-ear device.

In another aspect, a method includes providing an in-ear device to a pet anatomy; capturing accelerometer data and vital signs; controlling a virtual reality device or augmented reality device with acceleration or vital sign data from the in-ear device.

In another aspect, a method includes providing an in-ear device to a pet anatomy; capturing heart rate, EEG or ECG signal with the in-ear device; and determining pet intent with the in-ear device. The determined pet intent can be used to control an appliance, or open a door to the backyard for example.

In another aspect, a method includes providing an in-ear device to a pet anatomy; capturing heart rate, EEG/ECG signal or temperature data to detect biomarkers with the in-ear device; and predict health with the in-ear device data.

In another aspect, a method includes providing an in-ear device to a pet anatomy; capturing sounds from an advertisement, capturing vital signs associated with the advertisement; and customizing the advertisement to attract the user.

In another aspect, a method includes providing an in-ear device to a pet anatomy; capturing a command from a user, detecting pet emotion based on vital signs; and performing an action in response to the command and the detected pet emotion.

In another aspect, a method includes providing an in-ear device to a pet anatomy; capturing a command from a user, authenticating the pet based on a voiceprint or pet vital signs; and performing an action in response to the command.

In another aspect, a method includes providing an in-ear device to a pet anatomy; determine an audio response chart for a pet based on a plurality of environments (indoor or outdoor at restaurant, office, home, theater, party, concert, among others), determining a current environment, and updating the hearing aid parameters to optimize the amplifier response to the specific environment. The environment can be auto detected based on GPS position data or external data such as calendaring data or can be pet selected using voice command, for example. In another embodiment, a learning machine automatically selects an optimal set of hearing aid parameters based on ambient sound and other confirmatory data.

In another aspect, a user can remotely track a pet using a mobile app, and can issue a remote voice command which is wirelessly communicated to the pet's ear. If the pet does not respond, the system can increase the volume and replay the command. If the pet still does not respond, an electrical stimulus can be remotely sent. The stimulus can be a reward such as a FES massage or can be a punishment such as an electrical shock that causes a minor discomfort to the pet.

In another aspect, a user can remotely track a pet using a mobile app, and can issue a voice command to a suitable voice appliance/device such as the Amazon echo and the voice command is wirelessly communicated to the pet's ear. If the pet does not respond, the system can increase the volume and replay the command. If the pet still does not respond, an electrical stimulus can be remotely sent. The stimulus can be a reward such as a FES massage or can be a punishment such as an electrical shock that causes a minor discomfort to the pet.

In another aspect, a user can remotely track a pet using a mobile app, and can issue a remote command to turn on a display connected to the processor and mounted as part of the wearable housing. The display can be colored LEDs, or can be a flexible display such as flexible OLEDs.

In yet another aspect, flexible electronics is used as sensors and electronics as part of the housing.

Implementations of any of the above aspects may include one or more of the following:

detecting electrical potentials encephalography (EEG) or electrocardiogram (ECG) in the ear;

using a camera in the ear to detect ear health;

detecting blood flow with an in-ear sensor;

detecting with an in-ear sensor blood parameters including carboxyhemoglobin (HbCO), methemoglobin (HbMet) and total hemoglobin (Hbt);

detecting pressure based on a curvature of an ear drum;

detecting body temperature in the ear;

detecting one or more of: alpha rhythm, auditory steady-state response (ASSR), steady-state visual evoked potentials (SSVEP), visually evoked potential (VEP), visually evoked response (VER) and visually evoked cortical potential (VECP), cardiac activity, speech and breathing;

detecting alpha rhythm, auditory steady-state response (ASSR), steady-state visual evoked potentials (SSVEP), and visually evoked potential (VEP);

correlating EEG, ECG, speech and breathing to determine health;

correlating cardiac activity, speech and breathing;

determining pet health by detecting fluid in an ear structure, change in ear color, curvature of the ear structure;

determining one or more bio-markers from the vital signs and indicating pet health;

performing a 3D scan inside an ear canal;

matching predetermined points on the 3D scan to key points on a template and morphing the key points on the template to the predetermined points;

3D printing a model from the 3D scan and fabricating the in-ear device;

correlating genomic biomarkers for diseases to the vital signs from the in-ear device and applying a learning machine to use the vital signs from the in-ear device to predict disease conditions;

determining a fall based on accelerometer data, vital signs and sound; or

providing a user dashboard showing pet health data over a period of time and matching research data on the health signals.

Advantages of the above systems may include one or more of the following. The system increases attachment of owners with their pets for companionship, entertainment, fitness and mental wellbeing. The system helps humans to connect with their pets and track their daily activities. It enables activity tracking, monitors heart and respiratory rates along with rest patterns and calories burnt off by their dog or cats. These devices generate data regarding food intake of pets, which may be useful for owners to analyze their health and well-being. Wearable devices allow continuous monitoring and measurement of the biomechanical and physiological systems of the body along with body movement allowing the user to better understand pet behavior, or monitor health, etc., and enhance their engagement with the external environment. The system can transmit vital information about the pet health metrics to the veterinarians and owners. The combination of the wearable devices, mobile application, and the data analytics technology can be a main stream option for the value-based care of pets. The cloud-based data analytics services along with their products can help the veterinarians to diagnose and treat pets by providing valuable clinical information for real time decision making. Innovative technical solutions are provided to minimize power consumption by the wearable devices, and keep cost low. The system offers the convenience and remote control offered by interfacing the collar with a server, especially where a user can upload various geo-positional parameters, verbal cues and vocal commands, and also be able to track in real-time an animal's location. The system allows for remote programing of an animal collar and the retention of that programming so that re-programing of the animal collar is convenient and consistent in its operation.

For the pet training embodiment, the system reduces the human span required to train the pet, cat or dog, and along the way improves the relationship between the pet and its owner. Further, the owner is spared the unpleasant task of cleaning up after his or her pet. The owner can even reduce the time needed to walk a dog around the block to avoid soiling his or her home with pet wastes.

Other features and advantages of the present invention will become apparent from a reading of the following description as well as a study of the appended drawings.

### BRIEF DESCRIPTION OF THE DRAWINGS

A pet monitoring system incorporating the features of the preferred embodiment is depicted in the attached drawings which form a portion of the disclosure and wherein:

FIG. 1 is an exemplary process flow diagram showing part of the processing of the preferred embodiment;

FIGS. 2A-2C are exemplary process flow diagrams showing another portion of the processing with stimulus control of the pet;

FIG. 3A is an exemplary communication system infrastructure diagram showing a pet wearing the preferred embodiment and connected to various communication elements in which the collar operates;

FIG. 3B shows an exemplary wearable appliance in the ear (ITE);

FIG. 3C shows an exemplary pet hearing aid testing process;

FIG. 3D shows an exemplary learning system that identifies outliers;

FIG. 3E-3F shows an exemplary neural network and a deep learning system respectively for detecting pet activity from sensor monitoring;

FIG. 3G shows a data driven system for monitoring pet health;

FIG. 3H shows an exemplary Al based treatment consultation system for pets;

FIG. 4 is an exemplary side view of the preferred embodiment showing its shocking prongs and an external switch; and,

FIG. 5 is a diagram to show an exemplary house with outside potty area marked out.

### DESCRIPTION OF THE PREFERRED EMBODIMENTS

The detailed description set forth below in connection with the appended drawings is intended as a description of the preferred embodiment and is not intended to represent the only forms in which the present invention may be constructed and/or utilized. The description sets forth the functions and the sequence of steps for constructing and operating the preferred embodiment in connection with the illustrated embodiments. However, it is to be understood that the same or equivalent functions and sequences may be accomplished by different embodiments that are also intended to be encompassed within the spirit and scope of the invention.

Referring to the drawings for a better understanding of the function and structure of the various embodiments, a system for monitoring pets is disclosed. The system employs one or more sensors and applies machine learning to provide better care and training of the pets.

FIG. 1 shows an exemplary process to train the pet to avoid soiling the home interior:
Capture house location/coordinate information (202)
Define training boundardy (potty area and no-potty area) (204)
Detect odor indicative of pee or poop (206)
Provide negative stimulus when pet soils no-potty area (208)
Provide positive stimulus when pet uses potty area (210)

The stimulus can be electrical, light, audible, or any suitable indications of correctness or incorrectness that is provided as a regular feedback to cause the pet to be trained according to the owner's wish.

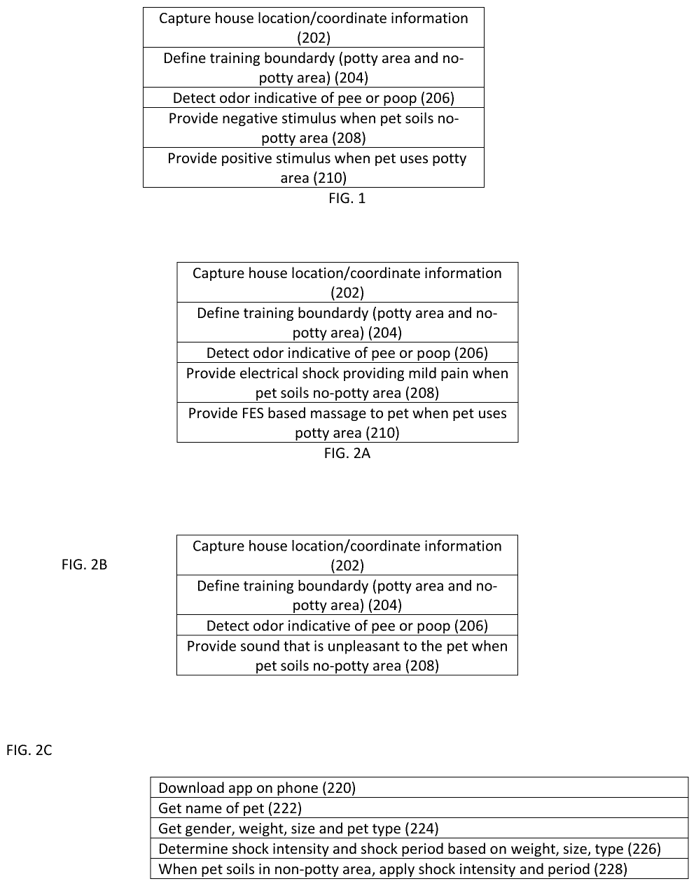

FIGS. 2A-2B are exemplary process flow diagrams showing another portion of the processing with stimulus control of the pet. FIG. 2A shows a process to apply electrical pulses as feedback:
Capture house location/coordinate information (202)
Define training boundardy (potty area and no-potty area) (204)
Detect odor indicative of pee or poop (206)
Provide electrical shock providing mild pain when pet soils no-potty area (208)
Provide functional electrical stimulation (FES) massage to pet when pet uses potty area (210)

One embodiment uses an electrical amplifier to provide Functional Electrical Stimulation (FES). The amplifier can provide a mild electric shock to the dog as a negative reinforcement, or can provide a message by pulsing the muscles to provide a pleasant response as a positive feedback. The functional electrical stimulation amplifier (b) injects electrical current into the cell (a). (c) The intact but dormant axon receives the stimulus and propagates an action potential to (d) the neuromuscular junction. (e) The corresponding muscle fibers contract and generate (f) muscle force. (g) A train of negative pulses is produced. (h) Depolarization occurs where negative current enters the axon at the “active” electrode indicated. FES uses a pulsating electrical current to direct the movement of muscles, tendons and ligaments through the replication of the natural motor nerve impulses. FES has been shown to be an extremely effective type of electrical stimulation for the rehabilitation of injuries, as well as for reducing the stress and strain of pet training. The treatment feels similar to a deep muscle massage. However, not only does the pet feel more relaxed during therapy, the deep muscle movement will release tension in areas that may have been constricted and sore for long periods of time.

In yet another embodiment, sound can be used to provide training reinforcement. As shown in FIG. 2B, the process is as follows:
Capture house location/coordinate information (202)
Define training boundardy (potty area and no-potty area) (204)
Detect odor indicative of pee or poop (206)
Provide sound that is unpleasant to the pet when pet soils no-potty area (208)

In FIG. 2B, high frequency audio that is imperceptible to human is used. Humans can hear sounds in a range from about 20 hertz to 23 kilohertz at the upper range of their hearing ability. The hearing range of dogs is almost double that. For example, a dog whistle, which sounds silent to humans, produces sounds in the 50-kilohertz range that dogs can hear. Dogs have better hearing than humans both because they can hear these high-frequency sounds, and they can hear sounds from farther away. This is because of the way their ears are designed. Their ears are made to cup and move sound in, similar to the way humans can put a hand up to their ear to hear better. Dogs can also move their ears around to hone in on sounds in different directions. Thus, the system uses high frequency sounds as feedback for the pet. The system can play high frequency sound that bothers the dog when it is defecating or peeing inside the area marked for the pet to avoid. Repetitive training with the sound will cause the dog to be conditioned to avoid the desired area.

FIG. 2C allows custom shock intensity/period to be applied based on the size and pet type, among others:
Download app on phone (220)
Get name of pet (222)
Get gender, weight, size and pet type (224)
Determine shock intensity and shock period based on weight, size, type (226)
When pet soils in non-potty area, apply shock intensity and period (228)

Thus, the process of FIG. 2C provides custom feedback so that the pet is not unduly harmed if it is too small, yet effective for the size of the pet.

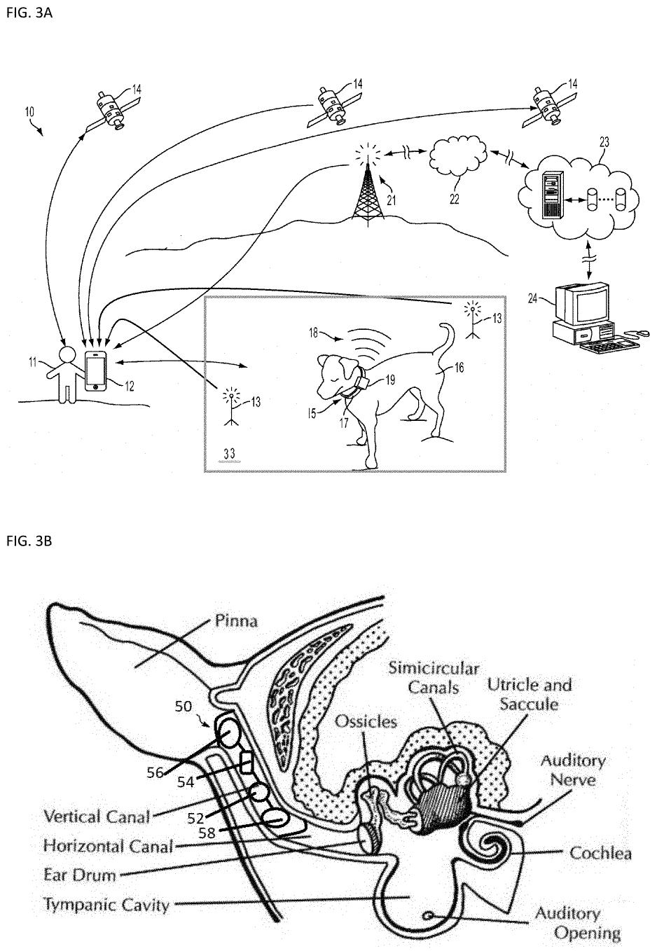

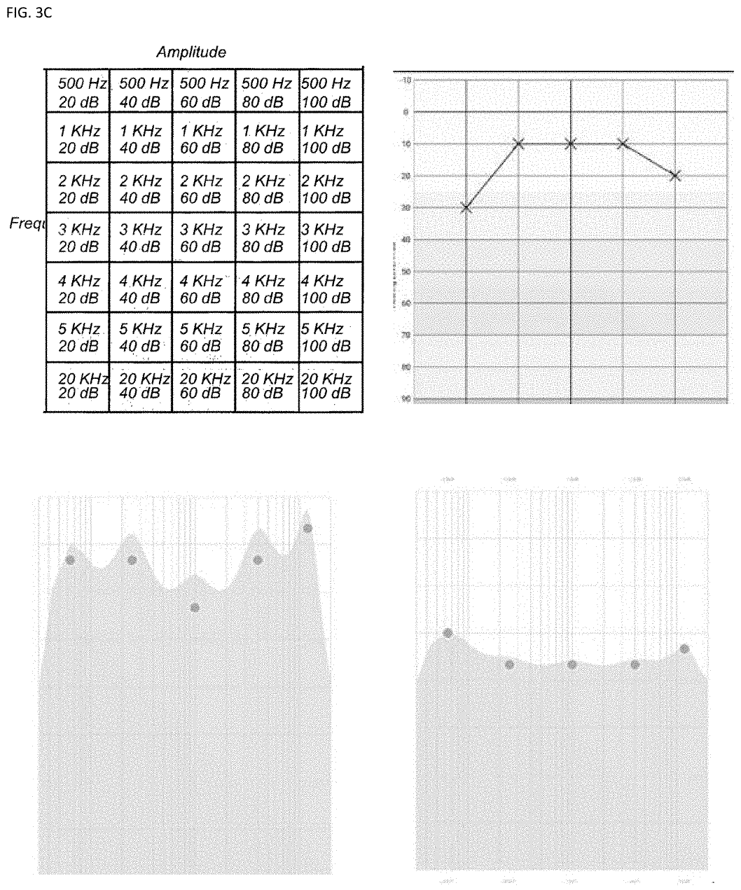

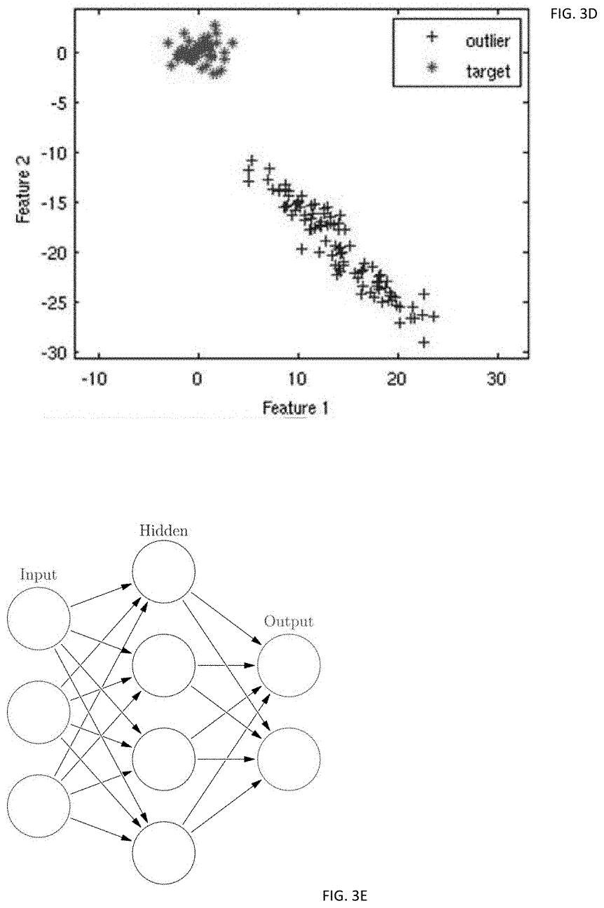

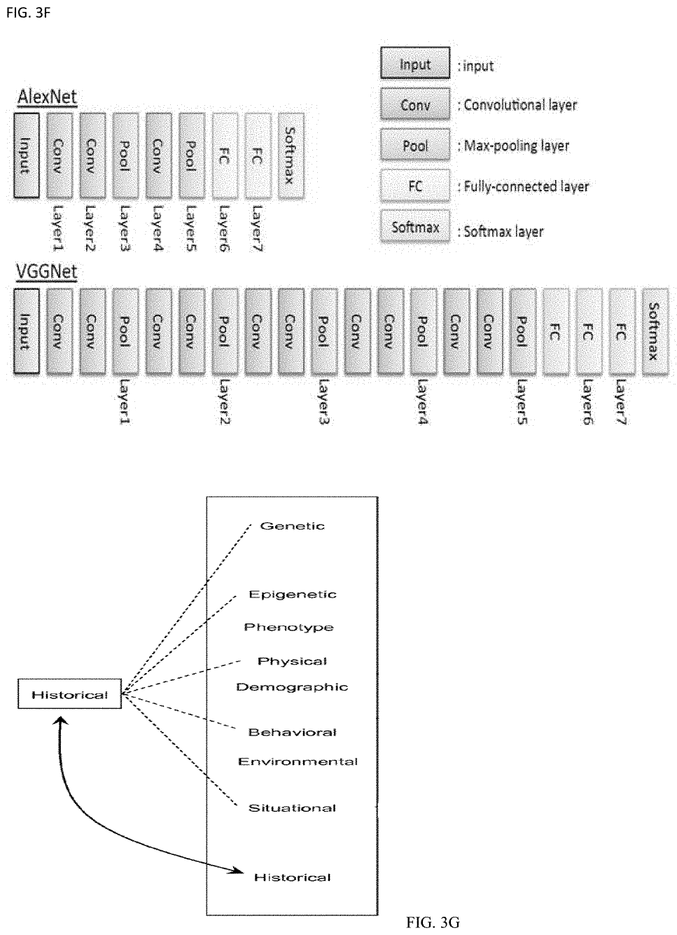

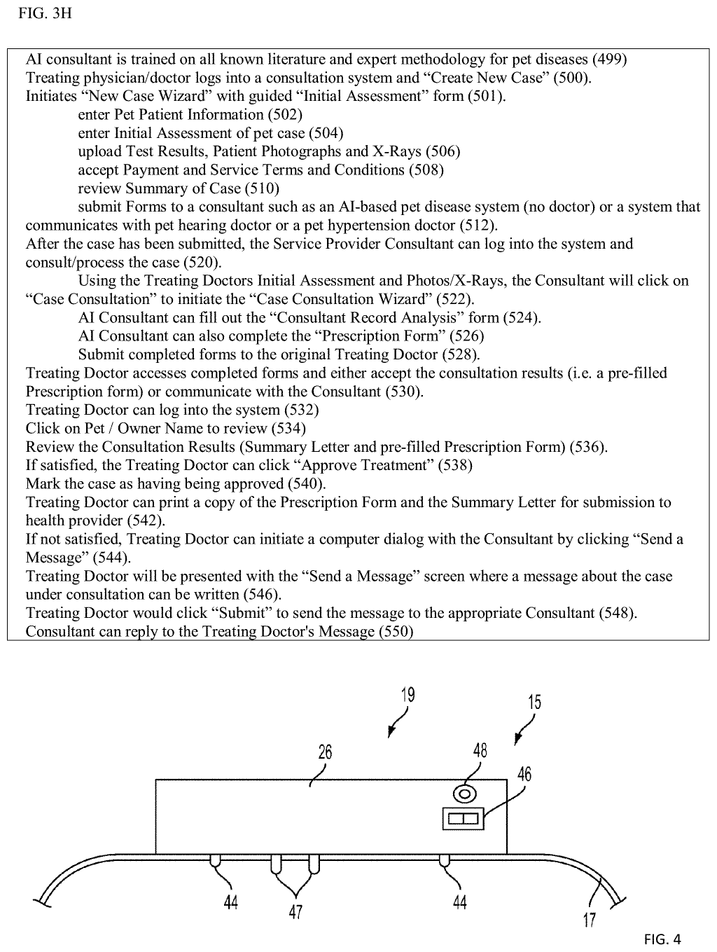

FIG. 3 shows a schematic view of the wireless infrastructure 10 utilized by the present preferred embodiment during typical use in a potty training scenario, for example. In this sample scenario, an individual 11 desires to train a pet 16 to train the pet to only pee outside of the home, and thus in this example, the dog should avoid peeing inside a home 33. In one embodiment, this is done by providing a gentle shock to negatively bias the dog from doing potty inside the confines of home 33. In another embodiment, the dog can be trained using negative enhancement such as sound that is painful to the dog but not hearable by humans. In another embodiment, the dog can be trained using positive enhancement such as sound that is pleasurable to the dog but not hearable by humans or by electrical pulses that massage the dog using FES, for example. The user initiates a software application on mobile device 12, which includes receivers capable of detecting signals originating from global positioning system (GPS) satellites 14, WiFi repeater/booster stations 13 inside the home to provide triangulation of positions for example, and one or more cell towers 21, as well as signal 18 originating from the electronics module 19 located on the dog's collar 15.

By connecting with the Internet 22 via WiFi, Bluetooth, or cell transmissions, the software application can access a geo-fence specification and the dog's geo-positional data for processing by a remote server, such as cloud server 23. The data contained on cloud server 23 can also be accessed and modified by remote computing device 24, such as a PC, via an Internet connection. External position accuracy is achieved by a network of GPS satellites that continuously transmit signals to the Earth; the data transmitted by these signals includes the precise time at which the signal was transmitted by the satellite. By noting the time at which the signal is received at a GPS receiver, a propagation time delay can be calculated. By multiplying the propagation time delay by the signal's speed of propagation, the GPS receiver can calculate the distance between the satellite and the receiver. This calculated distance is called a “pseudorange,” due to error introduced by the lack of synchronization between the receiver clock and GPS time, as well as atmospheric effects. Using signals from at least three satellites, at least three pseudoranges are calculated, and the position of the GPS receiver is determined through a geometrical triangulation calculation.

The mobile device 12 also has one or more sensors, including accelerometer(s) to monitor dog motion and activities of life. The accelerometer can capture motion and gait, as detailed below.

The device 12 also has heart rate circuit. A normal heart rate for dogs is 60-140 beats per minute, and for cats is 160-240 beats per minute. The software can detect sinus arrhythmia and rhythm of the pet's heartbeat, and the user can ask a vet to take a listen and make sure everything is okay.

The device 12 can monitor respiratory rate at rest and in motion. This is done by monitoring the breathing motion of the chest in one embodiment. At rest, a healthy dog takes between 12 and 24 breaths per minute, and a healthy cat takes between 20 and 30 breaths per minute. The system can count the number of times the chest expands/contracts. You can do this either by watching your pet or resting your hand on the ribs. The microphone can detect respiratory noise. Normal respiration should not make any noise, and should require very little effort. The system can adjust for a brachycephalic breed like a Pug, English Bulldog, Himalayan or Persian, a little snort from time to time can be expected.

The device 12 can monitor body temperature. A normal body temperature for dogs and cats is around 100.5 to 102.5° F. The system can keep a log of their normal numbers in the medical history including ALL medications they're taking and time to provide to the treating veterinarian.

Next, in-ear sensors are detailed. An ear site has the advantage of more quickly and more accurately reflecting oxygenation changes in the body's core as compared to peripheral site measurements, such as a fingertip. Variations in lobe size, shape and thickness and the general floppiness of the ear lobe render this site less suitable for central oxygen saturation measurements than the concha and the ear canal. Disclosed herein are various embodiments for obtaining noninvasive blood parameter measurements from concha 120 and ear canal 130 tissue sites.

In another embodiment, a hologram scanner can be used. The device for recording the spatial structure of at least one part of an ear canal or ear impression uses a holography unit with a light source and by means of which a hologram of the ear canal can be adjusted. A semitransparent disk in the ear is used for separating the light beam from the light source into an illumination beam and a reference beam. A sensor records an object beam, which is produced by reflection of the illumination beam onto the part of the ear canal, together with the reference beam. The hologram recording system can essentially be constructed within smaller dimensions than a conventional 3D scanner, which is based on the triangulation principle. The hologram recording system can be constructed within significantly smaller dimensions than the 3D scanner. The hologram sensor (CCD chip) does not require a front lens, since it does not record a mapping of an image, but instead interference patterns on its surface

One aspect of an ear sensor optically measures physiological parameters related to blood constituents by transmitting multiple wavelengths of light into a concha site and receiving the light after attenuation by pulsatile blood flow within the concha site. The ear sensor comprises a sensor body, a sensor connector and a sensor cable interconnecting the sensor body and the sensor connector. The sensor body comprises a base, legs and an optical assembly. The legs extend from the base to detector and emitter housings. An optical assembly has an emitter and a detector. The emitter is disposed in the emitter housing and the detector is disposed in the detector housing. The legs have an unflexed position with the emitter housing proximate the detector housing and a flexed position with the emitter housing distal the detector housing. The legs are moved to the flexed position so as to position the detector housing and emitter housing over opposite sides of a concha site. The legs are released to the unflexed position so that the concha site is grasped between the detector housing and emitter housing.

Pulse oximetry systems for measuring constituents of circulating blood can be used in many monitoring scenarios. A pulse oximetry system has an optical sensor applied to a pet, a monitor for processing sensor signals and displaying results and a pet cable electrically interconnecting the sensor and the monitor. A pulse oximetry sensor has light emitting diodes (LEDs), typically one emitting a red wavelength and one emitting an infrared (IR) wavelength, and a photodiode detector. The emitters and detector are in the ear insert, and the pet cable transmits drive signals to these emitters from the monitor. The emitters respond to the drive signals to transmit light into the fleshy tissue. The detector generates a signal responsive to the emitted light after attenuation by pulsatile blood flow within the fingertip. The pet cable transmits the detector signal to the monitor, which processes the signal to provide a numerical readout of physiological parameters such as oxygen saturation (SpO2) and pulse rate. Advanced physiological monitoring systems may incorporate pulse oximetry in addition to advanced features for the calculation and display of other blood parameters, such as carboxyhemoglobin (HbCO), methemoglobin (HbMet) and total hemoglobin (Hbt), as a few examples. In other embodiments, the device has physiological monitors and corresponding multiple wavelength optical sensors capable of measuring parameters in addition to SpO2, such as HbCO, HbMet and Hbt are described in at least U.S. patent application Ser. No. 12/056,179, filed Mar. 26, 2008, titled Multiple Wavelength Optical Sensor and U.S. patent application Ser. No. 11/366,208, filed Mar. 1, 2006, titled Noninvasive Multi-Parameter Pet Monitor, both incorporated by reference herein. Further, noninvasive blood parameter monitors and corresponding multiple wavelength optical sensors to sense SpO2, pulse rate, perfusion index (PI), signal quality (SiQ), pulse variability index (PVI), HbCO and HbMet among other parameters.

Heart pulse can be detected by measuring the dilation and constriction of tiny blood vessels in the ear canal. In one embodiment, the dilation measurement is done optically and in another embodiment, a micromechanical MEMS sensor is used. ECG sensor can be used where the electrode can detect a full and clinically valid electrocardiogram, which records the electrical activity of the heart.

One example embodiment uses the Samsung Bio-Processor which integrates multiple AFEs (Analog Front Ends) to measure diverse biometrics, including bioelectrical impedance analysis (BIA), photoplethysmogram (PPG), electrocardiogram (ECG), skin temperature and galvanic skin response (GSR) in a single chip solution. With integrated microcontroller unit (MCU), digital signal processor (DSP) and real-time clock (RTC), the Bio-Processor can monitor data in the ear with low power requirement.

Impact sensors, or accelerometers, measure in real time the force and even the number of impacts that players sustain. Data collected is sent wirelessly via Bluetooth to a dedicated monitor on the sidelines, while the impact prompts a visual light or audio alert to signal players, coaches, officials, and the training or medical staff of the team. One such sensor example is the ADXL377 from Analog Devices, a small, thin and low-power 3-axis accelerometer that measures acceleration from motion, shock, or vibration. It features a full-scale range of ±200 g, which would encompass the full range of impact acceleration in sports, which typically does not exceed 150 g's. When a post-impact individual is removed from a game and not allowed to return until cleared by a concussion-savvy healthcare professional, most will recover quickly. If the injury is undetected, however, and an athlete continues playing, concussion recovery often takes much longer. Thus, the system avoids problems from delayed or unidentified injury can include: Early dementia, Depression, Rapid brain aging, and Death. The cumulative effects of repetitive head impacts (RHI) increases the risk of long-term neuro-degenerative diseases, such as Parkinson's disease, Alzheimer's, Mild Cognitive Impairment, and ALS or Lou Gehrig's disease. The sensors' most important role is to alert to dangerous concussions for pets.

The device can use optical sensors for heart rate (HR) as a biomarker in heart failure (HF) both of diagnostic and prognostic values. HR is a determinant of myocardial oxygen demand, coronary blood flow, and myocardial performance and is central to the adaptation of cardiac output to metabolic needs. Increased HR can predict adverse outcome in the general population and in pets with chronic HF. Part of the ability of HR to predict risk is related to the forces driving it, namely, neurohormonal activation. HR relates to emotional arousal and reflects both sympathetic and parasympathetic nervous system activity. When measured at rest, HR relates to autonomic activity during a relaxing condition. HR reactivity is expressed as a change from resting or baseline that results after exposure to stimuli. These stress-regulating mechanisms prepare the body for fight or flight responses, and as such can explain individual differences to psychopathology. Thus, the device monitors HR as a biomarker of both diagnostic and prognostic values.

The HR output can be used to analyze heart-rate variability (HRV) (the time differences between one beat and the next) and HRV can be used to indicate the potential health benefits of food items. Reduced HRV is associated with the development of numerous conditions for example, diabetes, cardiovascular disease, inflammation, obesity and psychiatric disorders. Aspects of diet that are viewed as undesirable, for example high intakes of saturated or trans-fat and high glycaemic carbohydrates, have been found to reduce HRV. The consistent relationship between HRV, health and morbidity allows the system to use HRV as a biomarker when considering the influence of diet on mental and physical health. Further HRV can be used as a biomarker for aging.

In one embodiment, the system determines a dynamical marker of sino-atrial instability, termed heart rate fragmentation (HRF) and is used a dynamical biomarker of adverse cardiovascular events (CVEs). In healthy adults at rest and during sleep, the highest frequency at which the sino-atrial node (SAN) rate fluctuates varies between ˜0.15 and 0.40 Hz. These oscillations, referred to as respiratory sinus arrhythmia, are due to vagally-mediated coupling between the SAN and breathing. However, not all fluctuations in heart rate (HR) at or above the respiratory frequency are attributable to vagal tone modulation. Under pathologic conditions, an increased density of reversals in HR acceleration sign, not consistent with short-term parasympathetic control, can be observed.

The system captures ECG data as biomarkers for cardiac diseases such as myocardial infarction, cardiomyopathy, atrioventricular bundle branch block, and rhythm disorders. The ECG data is cleaned up, and the system extracts features by taking quantiles of the distributions of measures on ECGs, while commonly used characterizing feature is the mean. The system applies commonly used measurement variables on ECGs without preselection and use dimension reduction methods to identify biomarkers, which is useful when the number of input variables is large and no prior information is available on which ones are more important. Three frequently used classifiers are used on all features and to dimension-reduced features by PCA. The three methods are from classical to modern: stepwise discriminant analysis (SDA), SVM, and LASSO logistic regression.

In one embodiment, four types of features are considered as input variables for classification: T wave type, time span measurements, amplitude measurements, and the slopes of waveforms for features such as
(1) T Wave Type. The ECGPUWAVE function labels 6 types of T waves for each beat: Normal, Inverted, Positive Monophasic, Negative Monophasic, Biphasic Negative-Positive, and Biphasic Positive-Negative based on the T wave morphology. This is the only categorical variable considered.
(2) Time Span Measurements. Six commonly used time span measurements are considered: the length of the RR interval, PR interval, QT interval, P wave, QRS wave, and T wave.
(3) Amplitude Measurements. The amplitudes of P wave, R-peak, and T wave are used as input variables. To measure the P wave amplitude, we first estimate the baseline by taking the mean of the values in the PR segment, ST segment, and TP segment (from the end of the T wave to the start of the P wave of the next heartbeat), then subtract the maximum and minimum values of the P wave by the estimated baseline, and take the one with a bigger absolute value as the amplitude of P wave. Other amplitude measurements are obtained similarly.
(4) The Slopes of Waveforms. The slopes of waveforms are also considered to measure the dynamic features of a heartbeat. Each heartbeat is split into nine segments and the slope of the waveform in each segment is estimated by simple linear regression.

The device can include EEG sensors which measure a variety of EEG responses—alpha rhythm, ASSR, SSVEP and VEP—as well as multiple mechanical signals associated with cardiac activity, speech and breathing. EEG sensors can be used where electrodes provide low contact impedance with the skin over a prolonged period of time. A low impedance stretchable fabric is used as electrodes. The system captures various EEG paradigms: ASSR, steady-state visual evoked potential (SSVEP), transient response to visual stimulus (VEP), and alpha rhythm. The EEG sensors can predict and assess the fatigue based on the neural activity in the alpha band which is usually associated with the state of wakeful relaxation and manifests itself in the EEG oscillations in the 8-12 Hz frequency range, centered around 10 Hz. The loss of alpha rhythm is also one of the key features used by clinicians to define the onset of sleep. A mechanical transducer (electret condenser microphone) within its multimodal electro-mechanical sensor, which can be used as a reference for single-channel digital denoising of physiological signals such as jaw clenching and for removing real-world motion artifacts from ear-EEG. In one embodiment, a microphone at the tip of the earpiece facing towards the eardrum can directly capture acoustic energy traveling from the vocal chords via auditory tube to the ear canal. The output of such a microphone would be expected to provide better speech quality than the sealed microphone within the multimodal sensor.

The system can detect auditory steady-state response (ASSR) as a biomarker a type of ERP which can test the integrity of auditory pathways and the capacity of these pathways to generate synchronous activity at specific frequencies. ASSRs are elicited by temporally modulated auditory stimulation, such as a train of clicks with a fixed inter-click interval, or an amplitude modulated (AM) tone. After the onset of the stimulus, the EEG or MEG rapidly entrains to the frequency and phase of the stimulus. The ASSR is generated by activity within the auditory pathway. The ASSR for modulation frequencies up to 50 Hz is generated from the auditory cortex based on EEG. Higher frequencies of modulation (>80 Hz) are thought to originate from brainstem areas. The type of stimulus may also affect the region of activation within the auditory cortex. Amplitude modulated (AM) tones and click train stimuli are commonly used stimuli to evoke the ASSR.

The EEG sensor can be used as a brain-computer interface (BCI) and provides a direct communication pathway between the brain and the external world by translating signals from brain activities into machine codes or commands to control different types of external devices, such as a computer cursor, cellphone, home equipment or a wheelchair. SSVEP can be used in BCI due to high information transfer rate (ITR), little training and high reliability. The use of in-ear EEG acquisition makes BCI convenient, and highly efficient artifact removal techniques can be used to derive clean EEG signals.

The system can measure visually evoked potential (VEP), visually evoked response (VER) or visually evoked cortical potential (VECP). They refer to electrical potentials, initiated by brief visual stimuli, which are recorded from the scalp overlying visual cortex, VEP waveforms are extracted from the electro-encephalogram (EEG) by signal averaging. VEPs are used primarily to measure the functional integrity of the visual pathways from retina via the optic nerves to the visual cortex of the brain. VEPs better quantify functional integrity of the optic pathways than scanning techniques such as magnetic resonance imaging (MRI). Any abnormality that affects the visual pathways or visual cortex in the brain can affect the VEP. Examples are cortical blindness due to meningitis or anoxia, optic neuritis as a consequence of demyelination, optic atrophy, stroke, and compression of the optic pathways by tumors, amblyopia, and neurofibromatosis. In general, myelin plaques common in multiple sclerosis slow the speed of VEP wave peaks. Compression of the optic pathways such as from hydrocephalus or a tumor also reduces amplitude of wave peaks.

A bioimpedance (BI) sensor can be used to determine a biomarker of total body fluid content. The BIA is a noninvasive method for evaluation of body composition, easy to perform, and fast, reproducible, and economical and indicates nutritional status of pets by estimating the amount of lean body mass, fat mass, body water, and cell mass. The method also allows the assessment of pet's prognosis through the PA, which has been applied in pets with various diseases, including chronic liver disease. The phase angle varies according to the population and can be used for prognosis.

In another embodiment, the BI sensor can estimate glucose level. This is done by measuring the bioimpedance at various frequencies, where high frequency Bi is related to fluid volume of the body and low frequency BI is used to estimate the volume of extracellular fluid in the tissues.

The step of determining the amount of glucose can include comparing the measured impedance with a predetermined relationship between impedance and blood glucose level. In a particular embodiment, the step of determining the blood glucose level of a subject includes ascertaining the sum of a fraction of the magnitude of the measured impedance and a fraction of the phase of the measured impedance. The amount of blood glucose, in one embodiment, is determined according to the equation: Predicted glucose=(0.31)Magnitude+(0.24)Phase where the impedance is measured at 20 kHz. In certain embodiments, impedance is measured at a plurality of frequencies, and the method includes determining the ratio of one or more pairs of measurements and determining the amount of glucose in the body fluid includes comparing the determined ratio(s) with corresponding predetermined ratio(s), i.e., that have been previously correlated with directly measured glucose levels. In embodiments, the process includes measuring impedance at two frequencies and determining the amount of glucose further includes determining a predetermined index, the index including a ratio of first and second numbers obtained from first and second of the impedance measurements. The first and second numbers can include a component of said first and second impedance measurements, respectively. The first number can be the real part of the complex electrical impedance at the first frequency and the second number can be the magnitude of the complex electrical impedance at the second frequency. The first number can be the imaginary part of the complex electrical impedance at the first frequency and the second number can be the magnitude of the complex electrical impedance at the second frequency. The first number can be the magnitude of the complex electrical impedance at the first frequency and the second number can be the magnitude of the complex electrical impedance at the second frequency. In another embodiment, determining the amount of glucose further includes determining a predetermined index in which the index includes a difference between first and second numbers obtained from first and second of said impedance measurements. The first number can be the phase angle of the complex electrical impedance at the first frequency and said second number can be the phase angle of the complex electrical impedance at the second frequency.

The electrodes can be in operative connection with the processor programmed to determine the amount of glucose in the body fluid based upon the measured impedance. In certain embodiments, the processor wireless communicates with an insulin pump programmed to adjust the amount of insulin flow via the pump to the subject in response to the determined amount of glucose. The BIA electrodes can be spaced between about 0.2 mm and about 2 cm from each other.

In another aspect, the BI sensor provides non-invasive monitoring of glucose in a body fluid of a subject. The apparatus includes means for measuring impedance of skin tissue in response to a voltage applied thereto and a microprocessor operatively connected to the means for measuring impedance, for determining the amount of glucose in the body fluid based upon the impedance measurement(s). The means for measuring impedance of skin tissue can include a pair of spaced apart electrodes for electrically conductive contact with a skin surface. The microprocessor can be programmed to compare the measured impedance with a predetermined correlation between impedance and blood glucose level. The apparatus can include means for measuring impedance at a plurality of frequencies of the applied voltage and the program can include means for determining the ratio of one or more pairs of the impedance measurements and means for comparing the determined ratio(s) with corresponding predetermined ratio(s) to determine the amount of glucose in the body fluid.

In a particular embodiment, the apparatus includes means for calibrating the apparatus against a directly measured glucose level of a said subject. The apparatus can thus include means for inputting the value of the directly measured glucose level in conjunction with impedance measured about the same time, for use by the program to determine the blood glucose level of that subject at a later time based solely on subsequent impedance measurements.

One embodiment measures BI at 31 different frequencies logarithmically distributed in the range of 1 kHz to 1 Mhz (10 frequencies per decade). Another embodiment measures BI a t two of the frequencies: 20 and 500 kHz; and in the second set of experiments, 20 kHz only. It may be found in the future that there is a more optimal frequency or frequencies. It is quite possible, in a commercially acceptable instrument that impedance will be determined at at least two frequencies, rather than only one. For practical reasons of instrumentation, the upper frequency at which impedance is measured is likely to be about 500 kHz, but higher frequencies, even has high as 5 MHz or higher are possible and are considered to be within the scope of this preferred embodiment. Relationships may be established using data obtained at one, two or more frequencies.

One embodiment, specifically for determining glucose levels of a subject, includes a 2-pole BI measurement configuration that measures impedance at multiple frequencies, preferably two well spaced apart frequencies. The instrument includes a computer which also calculates the index or indices that correlate with blood glucose levels and determines the glucose levels based on the correlation(s). an artificial neural network to perform a non-linear regression.

In another embodiment, a BI sensor can estimate sugar content in human blood based on variation of dielectric permeability of a finger placed in the electrical field of transducer. The amount of sugar in human blood can also be estimate by changing the reactance of oscillating circuits included in the secondary circuits of high-frequency generator via direct action of human upon oscillating circuits elements. With this method, the amount of sugar in blood is determined based on variation of current in the secondary circuits of high-frequency generator. In another embodiment, a spectral analysis of high-frequency radiation reflected by human body or passing through the human body is conducted. The phase shift between direct and reflected (or transmitted) waves, which characterizes the reactive component of electrical impedance, represents a parameter to be measured by this method. The concentration of substances contained in the blood (in particular, glucose concentration) is determined based on measured parameters of phase spectrum. In another embodiment, glucose concentration is determined by this device based on measurement of human body region impedance at two frequencies, determining capacitive component of impedance and converting the obtained value of capacitive component into glucose concentration in pet's blood. Another embodiment measures impedance between two electrodes at a number of frequencies and deriving the value of glucose concentration on the basis of measured values. In another embodiment, the concentration of glucose in blood is determined based mathematical model.

The microphone can also detect respiration. Breathing creates turbulence within the airways, so that the turbulent airflow can be measured using a microphone placed externally on the upper chest at the suprasternal notch. The respiratory signals recorded inside the ear canal are weak, and are affected by motion artifacts arising from a significant movement of the earpiece inside the ear canal. A control loop involving knowledge of the degree of artifacts and total output power from the microphones can be used for denoising purposes from jaw movements. Denoising can be done for EEG, ECG, PPG waveforms.

An infrared sensor unit can detect temperature detection in conjunction with an optical identification of objects allows for more reliable identification of the objects, e.g. of the eardrum. Providing the device additionally with an infrared sensor unit, especially arranged centrically at the distal tip, allows for minimizing any risk of misdiagnosis.

In one implementation information relating to characteristics of the pet's tympanic cavity can be evaluated or processed. In this case the electronics includes a camera that detects serous or mucous fluid within the tympanic cavity can be an indicator of the eardrum itself, and can be an indicator of a pathologic condition in the middle ear. Within the ear canal, only behind the eardrum, such body fluid can be identified. Thus, evidence of any body fluid can provide evidence of the eardrum itself, as well as evidence of a pathologic condition, e.g. OME.

In a method according to the preferred embodiment, preferably, an intensity of illumination provided by the at least one light source is adjusted such that light emitted by the at least one light source is arranged for at least partially transilluminating the eardrum in such a way that it can be reflected at least partially by any object or body fluid within the subject's tympanic cavity arranged behind the eardrum. The preferred embodiment is based on the finding that translucent characteristics of the eardrum can be evaluated in order to distinguish between different objects within the ear canal, especially in order to identify the eardrum more reliably. Thereby, illumination can be adjusted such that tissue or hard bone confining the ear canal is overexposed, providing reflections (reflected radiation or light), especially reflections within a known spectrum, which can be ignored, i.e. automatically subtracted out. Such a method enables identification of the eardrum more reliably.

In particular, the degree of reddishness or reflectivity of light in the red spectral range can be determined at different illumination intensities. It can therefore be distinguished more reliably between light reflected by the eardrum itself, or by objects or fluids behind the eardrum, or by the mucosal covering the tympanic cavity wall. The reflectivity of light may be evaluated with respect to reflectivity within e.g. the green or blue spectral range. Typical spectral wavelength maxima are 450 nm (blue light), 550 nm (green light), and 600 nm (red light) for a respective (color) channel. The electronic imaging unit, e.g. comprising a color video camera, or any color sensitive sensor, may record images with respect to the red, green or blue spectral range, respectively. A logic unit may calculate, compare and normalize brightness values for each read, green and blue image, especially with respect to each separate pixel of the respective image. Such an evaluation may also facilitate medical characterization of the eardrum. In particular, the healthy eardrum is a thin, semitransparent membrane containing only few relatively small blood vessels. In contrast, an inflamed eardrum may exhibit thickening and/or increased vascularization. Also, any skin or tissue confining the ear canal as well as any mucosa in the middle ear may be heavily vascularized. In other words: The reflectivity in the different spectral ranges varies considerably between the different structures or objects as well as between healthy and inflamed tissue. Thus, referring to the spectral range enables more reliable differentiation between light reflected by the eardrum itself, or by objects or any fluid behind the eardrum, or by the tympanic cavity wall covered by mucosa.

Thereby, the risk of confounding any red (inflamed) section of the ear canal and the eardrum can be minimized. Also, the eardrum can be identified indirectly by identifying the tympanic cavity. In particular, any opaque fluid, especially amber fluid containing leukocytes and proteins, within the tympanic cavity may influence the spectrum of reflected light, depending on the intensity of illumination. At a relatively high intensity of illumination, the spectrum of reflected light will be typical for scattering in serous or mucous fluid containing particles like leukocytes, as light transmits the eardrum and is at least partially reflected by the opaque fluid. At a relatively low intensity of illumination, the spectrum of reflected light will be dominated by the eardrum itself, as a considerable fraction of the light does not transmit the eardrum, but is directly reflected by the eardrum. Thus, information relating to the tympanic cavity, especially more detailed color information, can facilitate identification of the eardrum as well as of pathologic conditions in the middle ear.

Transilluminating the eardrum can provide supplemental information with respect to the characteristics of the eardrum (e.g. the shape, especially a convexity of the eardrum), and/or with respect to the presence of any fluid within the tympanic cavity. Spectral patterns of reflected light which are typical for eardrum reflection and tympanic cavity reflection can be use to determine the area of interest as well as a physiologic or pathologic condition of the eardrum and the tympanic cavity, especially in conjunction with feedback controlled illumination.

Any fluid within the tympanic cavity evokes a higher degree of reflection than the physiologically present air. The fluid increases reflectance. In contrast, in case the tympanic cavity is filled with air, any light transilluminating the eardrum is only reflected with inferior intensity, as most of the light is absorbed within the tympanic cavity. In other words: transilluminating the eardrum and evaluating reflected light in dependence on the intensity of illumination can facilitate determining specific characteristics of the eardrum, e.g. an absolute degree of reflectivity in dependence on different wavelengths and intensities, providing more information or more certain information with respect to the type of tissue and its condition. Evaluating reflected light can comprise spectral analysis of translucent reflection, especially at different illumination intensities.

The degree of reflection in the red spectrum from the area of the eardrum may depend on the illumination level, i.e. the intensity of illumination. In particular, the red channel reflection can increase with increasing intensity of illumination. The higher the intensity of illumination, the higher the red channel reflection intensity. Also, it has been found that at relatively high intensities of illumination, not only the eardrum, but also any other tissue will reflect more light in the red spectrum. Therefore, on the one hand, providing a control or logic unit which is arranged for adjusting the intensity of illumination can facilitate identification of the eardrum. On the other hand, it can facilitate determining specific characteristics of the eardrum, e.g. an absolute degree of red channel reflection, such that the red channel reflection provides more information or more certain information with respect to the type of tissue and state of the tissue.

The degree of red channel reflection does not increase in the same manner with increasing intensity of illumination, depending on the presence of body fluid behind the eardrum. It has been found that in case there is body fluid within the tympanic cavity, with increasing intensity of illumination, the degree of red channel reflection does not increase as strongly as if the tympanic cavity was empty. Thus, based on the (absolute) degree of red channel reflection, the presence of fluid behind the eardrum can be evaluated. This may facilitate determination of pathologic conditions, e.g. OME.

The camera and process can identify pattern recognition of geometrical patterns, especially circular or ellipsoid shapes, or geometrical patterns characterizing the malleus bone, or further anatomical characteristics of the outer ear or the middle ear. Pattern recognition allows for more reliable identification of the eardrum. Pattern recognition can comprise recognition based on features and shapes such as the shape of e.g. the malleus, the malleus handle, the eardrum or specific portions of the eardrum such as the pasr flaccida or the fibrocartilagenous ring. In particular, pattern recognition may comprise edge detection and/or spectral analysis, especially shape detection of a circular or ellipsoid shape with an angular interruption at the malleus bone or pars flaccida.

In a method according to the preferred embodiment, preferably, the method further comprises calibrating a spectral sensitivity of the electronic imaging unit and/or calibrating color and/or brightness of the at least one light source. Calibration allows for more reliable identification of objects. It has been found that in case the light intensity is very high allowing for passing light through a healthy eardrum, which is semitransparent, a considerable amount of light within the red spectrum can be reflected by the tympanic cavity (especially due to illumination of red mucosa confining the middle ear). Thus, calibrating brightness or the intensity of emitted light enables more accurate evaluation of the (absolute) degree of red channel reflection and its source. In other words, spectral calibration of the imaging sensor in combination with spectral calibration of the illumination means allows for the evaluation of the tissue types and conditions.

Calibration can be carried out e.g. based on feedback illumination control with respect to different objects or different kinds of tissue, once the respective object or tissue has been identified. Thereby, spectral norm curves with respect to different light intensities provide further data based on which calibration can be carried out.

In one embodiment, FIG. 3B shows an earpiece 50 that has one or more sensors 52, a processor 54, a microphone 56, and a speaker 58. The earpiece 50 may be shaped and sized for an ear canal of a subject. The transducer 52 may be any of the previously discussed sensors (EEG, ECG, camera, temperature, pressure, among others). In general, the sensor 52 may be positioned within the earpiece at a position that, when the earpiece 50 is placed for use in the ear canal, corresponds to a location on a surface of the ear canal that exhibits a substantial shape change correlated to a musculoskeletal movement of the subject. The position depicted in FIG. 3B is provided by way of example only, and it will be understood that any position exhibiting substantial displacement may be used to position the sensor(s) 52 for use as contemplated herein. In one aspect, the sensor 52 may be positioned at a position that, when the earpiece is placed for use in the ear canal, corresponds to a location on a surface of the ear canal that exhibits a maximum surface displacement from a neutral position in response to the musculoskeletal movement of the subject. In another aspect, the transducer 52 may be positioned at a position that, when the earpiece is placed for use in the ear canal, corresponds to a location on a surface of the ear canal that exceeds an average surface displacement from a neutral position in response to the musculoskeletal movement of the subject. It will be understood that, while a single transducer 52 is depicted, a number of transducers may be included, which may detect different musculoskeletal movements, or may be coordinated to more accurately detect a single musculoskeletal movement.

The processor 54 may be coupled to the microphone 56, speaker 58, and sensor(s) 52, and may be configured to detect the musculoskeletal movement of the subject based upon a pressure change signal from the transducer 52, and to generate a predetermined control signal in response to the musculoskeletal movement. The predetermined control signal may, for example, be a mute signal for the earpiece, a volume change signal for the earpiece, or, where the earpiece is an earbud for an audio player (in which case the microphone 56 may optionally be omitted), a track change signal for the audio player coupled to the earpiece.

Power for the unit can be from a battery or scavenged from the environment using solar or temperature differential power generation. In one embodiment, a biological battery can be tapped. Located in the part of the ear called the cochlea, the battery chamber is divided by a membrane, some of whose cells are specialized to pump ions. An imbalance of potassium and sodium ions on opposite sides of the membrane, together with the particular arrangement of the pumps, creates an electrical voltage. A storage device receives charge that gradually builds up charge in a capacitor. The voltage of the biological battery fluctuates, for example one circuit needs between 40 seconds and four minutes to amass enough charge to power a radio. The frequency of the signal was thus itself an indication of the electrochemical properties of the inner ear.

To supplement the whisker antenna, the front and edge of the earpiece has 3D printed MIMO antennas for Wifi, Bluetooth, and 5G signals. The extension 39 further includes a microphone and camera at the tip to capture audio visual information to aid the user as an augmented reality system. The earpiece contains an inertial measurement unit (IMU) coupled to the intelligent earpiece. The IMU is configured to detect inertial measurement data that corresponds to a positioning, velocity, or acceleration of the intelligent earpiece. The earpiece also contains a global positioning system (GPS) unit coupled to the earpiece that is configured to detect location data corresponding to a location of the intelligent earpiece. At least one camera is coupled to the intelligent earpiece and is configured to detect image data corresponding to a surrounding environment of the intelligent guidance device.

Similar to humans, as dogs get old, they lose hearing acuity. Typically owners will often be able to get first inclination of hearing loss when their pet fails to respond to simple commands that they used to respond to. Also if it takes several calls or commands to get your pet to respond.

The system can run a BAER test. The hearing test known as the brainstem auditory evoked response (BAER) or brainstem auditory evoked potential (BAEP) detects electrical activity in the cochlea and auditory pathways in the brain in much the same way that an antenna detects radio or TV signals or an EKG detects electrical activity of the heart. The response waveform consists of a series of peaks numbered with Roman numerals: peak I is produced by the cochlear nerve and later peaks are produced within the brain.

In another embodiment, contrasting tones can be played to see if the dog can respond. As shown in FIG. 3C, the audiometer may generate pure tones at various frequencies between 125 Hz and 12,000 Hz that are representative of the frequency bands in which the tones are included. These tones may be transmitted through the headphones of the audiometer to the individual being tested. The intensity or volume of the pure tones is varied until the individual can just barely detect the presence of the tone. For each pure tone, the intensity of the tone at which the individual can just barely detect the presence of the tone is known as the individual's air conduction threshold of hearing. The collection of the thresholds of hearing at each of the various pure tone frequencies is known as an audiogram and may be presented in graphical form.

After audio equipment calibration, each frequency will be tested separately, at increasing levels. In one embodiment, the system starts with the lowest amplitude (quietest file at −5 dbHL for example) and stop when a user hearing threshold level has been reached. Files labelled 70 dBHL and above, are meant to detect severe hearing losses, and will play very loud for a normal hearing person. The equipment captures responses used to generate an audiogram which is a graph that shows the softest sounds a person can hear at different frequencies. It plots the threshold of hearing relative to an average ‘normal’ hearing. In this test, the ISO 389-7:2005 standard is used. Levels are expressed in deciBels Hearing Level (dBHL). The system saves the measurements and corresponding plots of Audiogram, Distortion, Time Analysis, Spectrogram, Audibility Spectrogram, 2-cc Curve, Occlusion Effects, and Feedback Analysis. A hearing aid prescription based on a selected fitting prescription formula/rational can be filled. The selected hearing aid can be adjusted and results analyzed and plotted with or without the involvement of the hearing-impaired individual. The system can optimize, objectively and subjectively, the performance of a selected hearing aid according to measured in-the-ear-canal probe response as a function of the selected signal model, hearing aid parameter set, the individual's measured hearing profile, and subjective responses to the presented audible signal. The system can determine the characteristics of a simulated monaural or binaural hearing aid system that produces natural sound perception and improved sound localization ability to the hearing impaired individual. This is accomplished by selecting a simulated hearing aid transfer function that produces, in conjunction with the face-plate transfer function, a combined transfer function that matches that of the unaided transfer function for each ear. The matching requirement typically involves frequency and phase responses. However, the magnitude response is expected to vary because most hearing impaired individuals require amplification to compensate for their hearing losses.

Based on the audiogram, amplifier parameters can be adjusted to improve hearing. In one embodiment for obtaining hearing enhancement fittings for a hearing aid device is described. In one embodiment, a plurality of audiograms is divided into one or more sets of audiograms. A representative audiogram is created for each set of audiograms. A hearing enhancement fitting is computed from each representative audiogram. A hearing aid device is programmed with one or more hearing enhancement fittings computed from each representative audiogram. In one embodiment, the one or more sets of audiograms may be subdivided into one or more subsets until a termination condition is satisfied. In one configuration, one or more audiograms may be filtered from the plurality of audiograms. For example, one or more audiograms may be filtered from the plurality of audiograms that exceed a specified fitting range for the hearing aid device. In one embodiment, a mean hearing threshold may be determined at each measured frequency of each audiogram within the plurality of audiograms. Prototype audiograms may be created from the mean hearing threshold. In addition, each prototype audiogram may be associated with a set of audiograms. In one configuration, an audiogram may be placed in the set of audiograms if the audiogram is similar to the prototype audiogram associated with the set. In one embodiment, the creation of a representative audiogram for each set of audiograms may include calculating a mean of each audiogram in a set of audiograms. When the threshold of hearing in each frequency band has been determined, this threshold may be used to estimate the amount of amplification, compression, and/or other adjustment that will be employed in the hearing aid device to compensate for the individual's loss of hearing. In the example of FIG. 6, the system will start at 500 Hz with progressing loudness before going to the next row at 1 kHz, 2, 3, 4, 5 and 20 kHz, respectively.

In one aspect, a method includes providing an in-ear device to a user anatomy; determine an audio response chart for a user based on a plurality of environments (restaurant, office, home, theater, party, concert, among others), determining a current environment, and updating the hearing aid parameters to optimize the amplifier response to the specific environment. The environment can be auto detected based on GPS position data or external data such as calendaring data or can be user selected using voice command, for example. In another embodiment, a learning machine automatically selects an optimal set of hearing aid parameters based on ambient sound and other confirmatory data.

A deep learning network can also be used to identify pet health. In embodiments that measure pet health with heart rate, BI, ECG, EEG, temperature, or other health parameters, if an outlier situation exists, the system can flag to the user to follow up as an unusual sustained variation from normal health parameters. While this approach may not identify exact causes of the variation, the user can seek help early. FIG. 3D shows an exemplary analysis with normal targets and outliers as warning labels. For example, a pet may be mostly healthy, but when it is sick, the information pops out as outliers from the usual data. Such outliers can be used to scrutinize and predict pet health. The data can be population based, namely that if a population spatially or temporally has the same symptoms, and upon checking with the medical hospitals or doctors to confirm the prediction, public health warnings can be generated. There are two main kinds of machine learning techniques: Supervised learning: in this approach, a training data sample with known relationships between variables is submitted iteratively to the learning algorithm until quantitative evidence (“error convergence”) indicates that it was able to find a solution which minimizes classification error. Several types of artificial neural networks work according to this principle; and Unsupervised learning: in this approach, the data sample is analyzed according to some statistical technique, such as multivariate regression analysis, principal components analysis, cluster analysis, etc., and automatic classification of the data objects into subclasses might be achieved, without the need for a training data set.

FIG. 3E shows a neural network for analyzing pet data, while FIG. 3F shows a deep learning system to analyze pet data. Medical prognosis can be used to predict the future evolution of disease on the basis of data extracted from known cases such as the prediction of mortality of pets admitted to the Intensive Care Unit, using physiological and pathological variables collected at admission. Medical diagnosis can be done, where ML is used to learn the relationship between several input variables (such as signs, symptoms, pet history, lab tests, images, etc.) and several output variables (the diagnosis categories). An example from my research: using symptoms related by pets with psychosis, an automatic classification system was devised to propose diagnoses of a particular disease. Medical therapeutic decisions can be done where ML is used to propose different therapies or pet management strategies, drugs, etc., for a given health condition or diagnosis. Example from my research: pets with different types of brain hematomas (internal bleeding) were used to train a neural network so that a precise indication for surgery was given after having learned the relationships between several input variables and the outcome. Signal or image analysis can be done, where ML is used to learn how features extracted from physiological signals (such as an EKG) or images (such as an x-ray, tomography, etc.) are associated to some diagnoses. ML can even be used to extract features from signals or images, for example, in the so-called “signal segmentation”. Example from my research: non-supervised algorithms were used to extract different image textures from brain MRIs (magnetic resonance imaging), such as bone, meninges, white matter, gray matter, vases, ventricles, etc., and then classifying automatically unknown images, painting each identified region with a different color. In another example large data sets containing multiple variables obtained from individuals in a given population (e.g., those living in a community, or who have a given health care plan, hospital, etc.), are used to train ML algorithms, so as to discover risk associations and predictions (for instance, what pets have a higher risk of emergency risk readmissions or complications from diabetes. Public health can apply ML to predict, for instance, when and where epidemics are going to happen in the future, such as food poisoning, infectious diseases, bouts of environmental diseases, and so on.

FIG. 3G shows an exemplary system to collect pet lifestyle and genetic data from various populations for subsequent prediction and recommendation to similarly situated users. The system collects attributes associated with individuals that co-occur (i.e., co-associate, co-aggregate) with attributes of interest, such as specific disorders, behaviors and traits. The system can identify combinations of attributes that predispose individuals toward having or developing specific disorders, behaviors and traits of interest, determining the level of predisposition of an individual towards such attributes, and revealing which attribute associations can be added or eliminated to effectively modify his or her lifestyle to avoid medical complications. Details captured can be used for improving individualized diagnoses, choosing the most effective therapeutic regimens, making beneficial lifestyle changes that prevent disease and promote health, and reducing associated health care expenditures. It is also desirable to determine those combinations of attributes that promote certain behaviors and traits such as success in sports, music, school, leadership, career and relationships. For example, the system captures information on epigenetic modifications that may be altered due to environmental conditions, life experiences and aging. Along with a collection of diverse nongenetic attributes including physical, behavioral, situational and historical attributes, the system can predict a predisposition of a user toward developing a specific attribute of interest. In addition to genetic and epigenetic attributes, which can be referred to collectively as pangenetic attributes, numerous other attributes likely influence the development of traits and disorders. These other attributes, which can be referred to collectively as non-pangenetic attributes, can be categorized individually as physical, behavioral, or situational attributes.

FIG. 3G displays one embodiment of the attribute categories and their interrelationships according to the one embodiment and illustrates that physical and behavioral attributes can be collectively equivalent to the broadest classical definition of phenotype, while situational attributes can be equivalent to those typically classified as environmental. In one embodiment, historical attributes can be viewed as a separate category containing a mixture of genetic, epigenetic, physical, behavioral and situational attributes that occurred in the past. Alternatively, historical attributes can be integrated within the genetic, epigenetic, physical, behavioral and situational categories provided they are made readily distinguishable from those attributes that describe the individual's current state. In one embodiment, the historical nature of an attribute is accounted for via a time stamp or other time-based marker associated with the attribute. As such, there are no explicit historical attributes, but through use of time stamping, the time associated with the attribute can be used to make a determination as to whether the attribute is occurring in what would be considered the present, or if it has occurred in the past. Traditional demographic factors are typically a small subset of attributes derived from the phenotype and environmental categories and can be therefore represented within the physical, behavioral and situational categories.

Since the system captures information from various diverse populations, the data can be mined to discover combinations of attributes regardless of number or type, in a population of any size, that cause predisposition to an attribute of interest. The ability to accurately detect predisposing attribute combinations naturally benefits from being supplied with datasets representing large numbers of individuals and having a large number and variety of attributes for each. Nevertheless, the one embodiment will function properly with a minimal number of individuals and attributes. One embodiment of the one embodiment can be used to detect not only attributes that have a direct (causal) effect on an attribute of interest, but also those attributes that do not have a direct effect such as instrumental variables (i.e., correlative attributes), which are attributes that correlate with and can be used to predict predisposition for the attribute of interest but are not causal. For simplicity of terminology, both types of attributes are referred to herein as predisposing attributes, or simply attributes, that contribute toward predisposition toward the attribute of interest, regardless of whether the contribution or correlation is direct or indirect.

FIG. 3F shows a deep learning machine using deep convolutionary neural networks for detecting genetic based drug-drug interaction. One embodiment uses an AlexNet: 8-layer architecture, while another embodiment uses a VGGNet: 16-layer architecture (each pooling layer and last 2 FC layers are applied as feature vector). In one embodiment for drugs, the indications of use and other drugs used capture most of many important covariates. One embodiment access data from SIDER (a text-mined database of drug package inserts), the Offsides database that contains information complementary to that found in SIDER and improves the prediction of protein targets and drug indications, and the Twosides database of mined putative DDIs also lists predicted adverse events, all available at the http://PharmGKB.org Web site.

The system of FIG. 3F receives data on adverse events strongly associated with indications for which the indication and the adverse event have a known causative relationship. A drug-event association is synthetic if it has a tight reporting correlation with the indication (p≥0.1) and a high relative reporting (RR) association score (RR≥2). Drugs reported frequently with these indications were 80.0 (95% Cl, 14.2 to 3132.8; P<0.0001, Fisher's exact test) times as likely to have synthetic associations with indication events. Disease indications are a significant source of synthetic associations. The more disproportionately a drug is reported with an indication (x axis), the more likely that drug will be synthetically associated. For example, adverse events strongly associated with drugs are retrieved from the drug's package insert. These drug-event pairs represent a set of known strong positive associations.

Adverse events related to sex and race are also analyzed. For example, for physiological reasons, certain events predominantly occur in males (for example, penile swelling and azoospermia). Drugs that are disproportionately reported as causing adverse events in males were more likely to be synthetically associated with these events. Similarly, adverse events that predominantly occur in either relatively young or relatively old pets are analyzed.

“Off-label” adverse event data is also analyzed, and off-label uses refer to any drug effect not already listed on the drug's package insert. Polypharmacy side effects for pairs of drugs (Twosides) are also analyzed. These associations are limited to only those that cannot be clearly attributed to either drug alone (that is, those associations covered in Offsides). The database contains a significant association for which the drug pair has a higher side-effect association score, determined using the proportional reporting ratio (PRR), than those of the individual drugs alone. The system determines pairwise similarity metrics between all drugs in the Offsides and SIDER databases. The system can predict shared protein targets using drug-effect similarities. The side-effect similarity score between two drugs is linearly related to the number of targets that those drugs share.

The system can determine relationships between the proportion of shared indications between a pair of drugs and the similarity of their side-effect profiles in Offsides. The system can use side-effect profiles to suggest new uses for old drugs. While the preferred system predicts existing therapeutic indications of known drugs, the system can recommend drug repurposing using drug-effect similarities in Offsides.

Corroboration of class-wide interaction effects with EMRs. The system can identify DDIs shared by an entire drug class. The class-class interaction analysis generates putative drug class interactions. The system analyzes laboratory reports commonly recorded in EMRs that may be used as markers of these class-specific DDIs.

In one embodiment, the knowledge-based repository may aggregate relevant clinical and/or behavioral knowledge from one or more sources. In an embodiment, one or more clinical and/or behavioral experts may manually specify the required knowledge. In another embodiment, an ontology-based approach may be used. For example, the knowledge-based repository may leverage the semantic web using techniques, such as statistical relational learning (SRL). SRL may expand probabilistic reasoning to complex relational domains, such as the semantic web. The SRL may achieve this using a combination of representational formalisms (e.g., logic and/or frame based systems with probabilistic models). For example, the SRL may employ Bayesian logic or Markov logic. For example, if there are two objects—‘asian male’ and ‘smartness’, they may be connected using the relationship ‘Asian males are smart’. This relationship may be given a weight (e.g., 0.3). This relationship may vary from time to time (populations trend over years/decades). By leveraging the knowledge in the semantic web (e.g., all references and discussions on the web where ‘blonde’ and ‘smartness’ are used and associated) the degree of relationship may be interpreted from the sentiment of such references (e.g., positive sentiment: TRUE; negative sentiment: FALSE). Such sentiments and the volume of discussions may then be transformed into weights. Accordingly, although the system originally assigned a weight of 0.3, based on information from semantic web about Asian males and smartness, may be revised to 0.9.

In an embodiment, Markov logic may be applied to the semantic web using two objects: first-order formulae and their weights. The formulae may be acquired based on the semantics of the semantic web languages. In one embodiment, the SRL may acquire the weights based on probability values specified in ontologies. In another embodiment, where the ontologies contain individuals, the individuals can be used to learn weights by generative learning. In some embodiments, the SRL may learn the weights by matching and analyzing a predefined corpus of relevant objects and/or textual resources. These techniques may be used to not only to obtain first-order waited formulae for clinical parameters, but also general information. This information may then be used when making inferences.

For example, if the first order logic is ‘obesity causes hypertension, there are two objects involved: obesity and hypertension. If data on pets with obesity and as to whether they were diagnosed with diabetes or not is available, then the weights for this relationship may be learnt from the data. This may be extended to non-clinical examples such as person's mood, beliefs etc.

The pattern recognizer may use the temporal dimension of data to learn representations. The pattern recognizer may include a pattern storage system that exploits hierarchy and analytical abilities using a hierarchical network of nodes. The nodes may operate on the input patterns one at a time. For every input pattern, the node may provide one of three operations: 1. Storing patterns, 2. Learning transition probabilities, and 3. Context specific grouping.

A node may have a memory that stores patterns within the field of view. This memory may permanently store patterns and give each pattern a distinct label (e.g. a pattern number). Patterns that occur in the input field of view of the node may be compared with patterns that are already stored in the memory. If an identical pattern is not in the memory, then the input pattern may be added to the memory and given a distinct pattern number. The pattern number may be arbitrarily assigned and may not reflect any properties of the pattern. In one embodiment, the pattern number may be encoded with one or more properties of the pattern.

In one embodiment, patterns may be stored in a node as rows of a matrix. In such an embodiment, C may represent a pattern memory matrix. In the pattern memory matrix, each row of C may be a different pattern. These different patterns may be referred to as C-1, C-2, etc., depending on the row in which the pattern is stored.

The nodes may construct and maintain a Markov graph. The Markov graph may include vertices that correspond to the store patterns. Each vertex may include a label of the pattern that it represents. As new patterns are added to the memory contents, the system may add new vertices to the Markov graph. The system may also create a link between to vertices to represent the number of transition events between the patterns corresponding to the vertices. For example, when an input pattern is followed by another input pattern j for the first time, a link may be introduced between the vertices i and j and the number of transition events on that link may be set to 1. System may then increment the number of transition counts on the link from i and j whenever a pattern from i to pattern j is observed. The system may normalize the Markov graph such that the links estimate the probability of a transaction. Normalization may be achieved by dividing the number of transition events on the outgoing links of each vertex by the total number of transition events from the vertex. This may be done for all vertices to obtain a normalized Markov graph. When normalization is completed, the sum of the transition probabilities for each node should add to 1. The system may update the Markov graph continuously to reflect new probability estimates.

The system may also perform context-specific grouping. To achieve this, the system may partition a set of vertices of the Markov graph into a set of temporal groups. Each temporal group may be a subset of that set of vertices of the Markov graph. The partitioning may be performed such that the vertices of the same temporal group are highly likely to follow one another.

The node may use Hierarchical Clustering (HC) to for the temporal groups. The HC algorithm may take a set of pattern labels and their pair-wise similarity measurements as inputs to produce clusters of pattern labels. The system may cluster the pattern labels such that patterns in the same cluster are similar to each other.

As data is fed into the pattern recognizer, the transition probabilities for each pattern and pattern-of-patterns may be updated based on the Markov graph. This may be achieved by updating the constructed transition probability matrix. This may be done for each pattern in every category of patterns. Those with higher probabilities may be chosen and placed in a separate column in the database called a prediction list.

Logical relationships among the patterns may be manually defined based on the clinical relevance. This relationship is specified as first-order logic predicates along with probabilities. These probabilities may be called beliefs. In one embodiment, a Bayesian Belief Network (BBN) may be used to make predictions using these beliefs. The BBN may be used to obtain the probability of each occurrence. These logical relationships may also be based on predicates stored the knowledge base.

The pattern recognizer may also perform optimization for the predictions. In one embodiment, this may be accomplished by comparing the predicted probability for a relationship with its actual occurrence. Then, the difference between the two may be calculated. This may be done for p occurrences of the logic and fed into a K-means clustering algorithm to plot the Euclidean distance between the points. A centroid may be obtained by the algorithm, forming the optimal increment to the difference. This increment may then be added to the (p+1)th occurrence. Then, the process may be repeated. This may be done until the pattern recognizer predicts logical relationships up to a specified accuracy threshold. Then, the results may be considered optimal.

When a node is at the first level of the hierarchy, its input may come directly from the data source, or after some preprocessing. The input to a node at a higher-level may be the concatenation of the outputs of the nodes that are directly connected to it from a lower level. Patterns in higher-level nodes may represent particular coincidences of their groups of children. This input may be obtained as a probability distribution function (PDF). From this PDF, the probability that a particular group is active may be calculated as the probability of the pattern that has the maximum likelihood among all the patterns belonging to that group.

The system can use an expert system that can assess hypertension in according with the guidelines. In addition, the expert system can use diagnostic information and apply the following rules to assess hypertension:

Hemoglobin/hematocrit: Assesses relationship of cells to fluid volume (viscosity) and may indicate risk factors such as hypercoagulability, anemia.

Blood urea nitrogen (BUN)/creatinine: Provides information about renal perfusion/function.

Glucose: Hyperglycemia (diabetes mellitus is a precipitator of hypertension) may result from elevated catecholamine levels (increases hypertension).

Serum potassium: Hypokalemia may indicate the presence of primary aldosteronism (cause) or be a side effect of diuretic-therapy.

Serum calcium: Imbalance may contribute to hypertension.

Lipid panel (total lipids, high-density lipoprotein [HDL], low-density lipoprotein [LDL], cholesterol, triglycerides, phospholipids): Elevated level may indicate predisposition for/presence of atheromatous plaques.

Thyroid studies: Hyperthyroidism may lead or contribute to vasoconstriction and hypertension.

Serum/urine aldosterone level: May be done to assess for primary aldosteronism (cause).

Urinalysis: May show blood, protein, or white blood cells; or glucose suggests renal dysfunction and/or presence of diabetes.

Creatinine clearance: May be reduced, reflecting renal damage.

Urine vanillylmandelic acid (VMA) (catecholamine metabolite): Elevation may indicate presence of pheochromocytoma (cause); 24-hour urine VMA may be done for assessment of pheochromocytoma if hypertension is intermittent.

Uric acid: Hyperuricemia has been implicated as a risk factor for the development of hypertension.

Renin: Elevated in renovascular and malignant hypertension, salt-wasting disorders.

Urine steroids: Elevation may indicate hyperadrenalism, pheochromocytoma, pituitary dysfunction, Cushing's syndrome.

Intravenous pyelogram (IVP): May identify cause of secondary hypertension, e.g., renal parenchymal disease, renal/ureteral-calculi.

Kidney and renography nuclear scan: Evaluates renal status (TOD).

Excretory urography: May reveal renal atrophy, indicating chronic renal disease.

Chest x-ray: May demonstrate obstructing calcification in valve areas; deposits in and/or notching of aorta; cardiac enlargement.

Computed tomography (CT) scan: Assesses for cerebral tumor, CVA, or encephalopathy or to rule out pheochromocytoma.

Electrocardiogram (ECG): May demonstrate enlarged heart, strain patterns, conduction disturbances. Note: Broad, notched P wave is one of the earliest signs of hypertensive heart disease.

The system may also be adaptive. In one embodiment, every level has a capability to obtain feedback information from higher levels. This feedback may inform about certain characteristics of information transmitted bottom-up through the network. Such a closed loop may be used to optimize each level's accuracy of inference as well as transmit more relevant information from the next instance.

The system may learn and correct its operational efficiency over time. This process is known as the maturity process of the system. The maturity process may include one or more of the following flow of steps:

a. Tracking patterns of input data and identifying predefined patterns (e.g. if the same pattern was observed several times earlier, the pattern would have already taken certain paths in the hierarchical node structure).

b. Scanning the possible data, other patterns (collectively called Input Sets (IS)) required for those paths. It also may check for any feedback that has come from higher levels of hierarchy. This feedback may be either positive or negative (e.g., the relevance of the information transmitted to the inferences at higher levels). Accordingly, the system may decide whether to send this pattern higher up the levels or not, and if so whether it should it send through a different path.

c. Checking for frequently required ISs and pick the top ‘F’ percentile of them.

d. Ensuring it keeps this data ready.

In one embodiment, information used at every node may act as agents reporting on the status of a hierarchical network. These agents are referred to as Information Entities (In En). In En may provide insight about the respective inference operation, the input, and the result which collectively is called knowledge.

This knowledge may be different from the KB. For example, the above described knowledge may include the dynamic creation of insights by the system based on its inference, whereas the KB may act as a reference for inference and/or analysis operations. The latter being an input to inference while the former is a product of inference. When this knowledge is subscribed to by a consumer (e.g. administering system or another node in a different layer) it is called “Knowledge-as-a-Service (KaaS)”

One embodiment processes behavior models are classified into four categories as follows:

a. Outcome-based;

b. Behavior-based;

c. Determinant-based; and

d. Intervention-based.

One or more of the following rules of thumb may be applied during behavioral modeling:

One or more interventions affect determinants;

One or more determinants affect behavior; and

One or more behaviors affect outcome.

A behavior is defined to be a characteristic of an individual or a group towards certain aspects of their life such as health, social interactions, etc. These characteristics are displayed as their attitude towards such aspects. In analytical terms, a behavior can be considered similar to a habit. Hence, a behavior may be observed for a given data from a pet. An example of a behavior is dietary habits.

Determinants may include causal factors for behaviors. They either cause someone to exhibit the same behavior or cause behavior change. Certain determinants are quantitative but most are qualitative. Examples include one's perception about a food, their beliefs, their confidence levels, etc.

Interventions are actions that affect determinants. Indirectly they influence behaviors and hence outcomes. System may get both primary and secondary sources of data. Primary sources may be directly reported by the end-user and AU. Secondary data may be collected from sensors such as their mobile phones, cameras, microphone, as well as those collected from general sources such as the semantic web.

These data sources may inform the system about the respective interventions. For example, to influence a determinant called forgetfulness which relates to a behavior called medication, the system sends a reminder at an appropriate time, as the intervention. Then, feedback is obtained whether the user took the medication or not. This helps the system in confirming if the intervention was effective.

The system may track a user's interactions and request feedback about their experience through assessments. The system may use this information as part of behavioral modeling to determine if the user interface and the content delivery mechanism have a significant effect on behavior change with the user. The system may use this information to optimize its user interface to make it more personalized over time to best suit the users, as well as to best suit the desired outcome.

The system also may accommodate data obtained directly from the end-user, such as assessments, surveys, etc. This enables users to share their views on interventions, their effectiveness, possible causes, etc. The system's understanding of the same aspects is obtained by way of analysis and service by the pattern recognizer.

Both system-perceived and end user-perceived measures of behavioral factors may be used in a process called Perception Scoring (PS). In this process, hybrid scores may be designed to accommodate both above mentioned aspects of behavioral factors. Belief is the measure of confidence the system has, when communicating or inferring on information. Initially higher beliefs may be set for user-perceived measures.

Over time, as the system finds increasing patterns as well as obtains feedback in pattern recognizer, the system may evaluate the effectiveness of intervention(s). If the system triggers an intervention based on user-perceived measures and it doesn't have significant effect on the behavior change, the system may then start reducing its belief for user-perceived measures and instead will increase its belief for system-perceived ones. In other words, the system starts believing less in the user and starts believing more in itself. Eventually this reaches a stage where system can understand end-users and their behavioral health better than end-users themselves. When perception scoring is done for each intervention, it may result in a score called Intervention Effectiveness Score (IES).

Perception scoring may be done for both end-users as well as AU. Such scores may be included as part of behavior models during cause-effect analysis.

Causes may be mapped with interventions, determinants, and behavior respectively in order of the relevance. Mapping causes with interventions helps in back-tracking the respective AU for that cause. In simple terms, it may help in identifying whose actions have had a pronounced effect on the end-user's outcome, by how much and using which intervention. This is very useful in identifying AUs who are very effective with specific interventions as well as during certain event context. Accordingly, they may be provided a score called Associated User Influence Score. This encompasses information for a given end-user, considering all interventions and possible contexts relevant to the user's case.

The system may construct one or plans including one or more interventions based on analysis performed, and may be implemented. For example, the system may analyze eligibility of an intervention for a given scenario, evaluating eligibility of two or more interventions based on combinatorial effect, prioritizing interventions to be applied, based on occurrence of patterns (from pattern recognizer), and/or submitting an intervention plan to the user or doctor in a format readily usable for execution.

This system may rely on the cause-effect analysis for its planning operations. A plan consists of interventions and a respective implementation schedule. Every plan may have several versions based on the users involved in it. For example, the system may have a separate version for the physician as compared to a pet. They will in turn do the task and report back to the system. This can be done either directly or the system may indirectly find it based on whether a desired outcome with the end user was observed or not.

The methodology may be predefined by an analyst. For every cause, which can be an intervention(s), determinant(s), behavior(s) or combinations of the same, the analyst may specify one or more remedial actions. This may be specified from the causal perspective and not the contextual perspective.

Accordingly, the system may send a variety of data and information to pattern recognizer and other services, as feedback, for these services to understand about the users. This understanding may affect their next set of plans which in turn becomes an infinite cyclic system where system affects the users while getting affected by them at the same time. Such a system is called a reflexive-feedback enabled system. The system may user both positive and negative reflexive-feedback, though the negative feedback aspect may predominantly be used for identifying gaps that the system needs to address.

The system may provide information, such as one or more newly identified patterns, to an analyst (e.g., clinical analyst or doctor). In the use case, the doctor may be presented with one or more notifications to address the relationship between carbohydrates and the medication that the pet is taking.

One embodiment of the system operation includes receiving feedback relating to the plan, and revising the plan based on the feedback; the feedback being one or more pet behaviors that occur after the plan; the revised plan including one or more additional interventions selected based on the feedback; the one or more pet behaviors that occur after the plan include a behavior transition; determining one or more persons to associate with the identified intervention; automatically revising probabilities from the collected information; storing the revised probabilities, wherein the revised probabilities are used to determine the plan; and/or automatically make one or more inferences based on machine learning using one or more of the clinical information, behavior information, or personal information.

The system can track health issues such as hypertension in dogs and cats, for example. More commonly referred to as high blood pressure, hypertension occurs when the dog's arterial blood pressure is continually higher than normal. When it is caused by another disease, it is called secondary hypertension; primary hypertension, meanwhile, refers to when it actually is the disease. Hypertension may affect many of the dog's body systems, including heart, kidneys, eyes, and the nervous system. The methods and systems disclosed herein may rely on one or more algorithm(s) to analyze one or more of the described metrics. The algorithm(s) may comprise analysis of data reported in real-time, and may also analyze data reported in real-time in conjunction with auxiliary data stored in a hypertension management database. Such auxiliary data may comprise, for example, historical pet data such as previously-reported hypertension metrics (e.g., hypertension scores, functionality scores, medication use), personal medical history, and/or family medical history. In some embodiments, for example, the auxiliary data includes at least one set of hypertension metrics previously reported and stored for a pet. In some embodiments, the auxiliary data includes a pet profile such as, e.g., the pet profile described above. Auxiliary data may also include statistical data, such as hypertension metrics pooled for a plurality of pets within a similar group or subgroup. Further, auxiliary data may include clinical guidelines such as guidelines relating to hypertension management, including evidence-based clinical practice guidelines on the management of acute and/or chronic hypertension or other chronic conditions.

Analysis of a set of hypertension metrics according to the present disclosure may allow for calibration of the level, degree, and/or quality of hypertension experienced by providing greater context to pet-reported data. For example, associating a hypertension score of 7 out of 10 with high functionality for a first pet, and the same score with low functionality for a second pet may indicate a relatively greater debilitating effect of hypertension on the second pet than the first pet. Further, a high hypertension score reported by a pet taking a particular medication such as opioid analgesics may indicate a need to adjust the pet's treatment plan. Further, the methods and systems disclosed herein may provide a means of assessing relative changes in a pet's distress due to hypertension over time. For example, a hypertension score of 5 out of 10 for a pet who previously reported consistently lower hypertension scores, e.g., 1 out of 10, may indicate a serious issue requiring immediate medical attention.

Any combination(s) of hypertension metrics may be used for analysis in the systems and methods disclosed. In some embodiments, for example, the set of hypertension metrics comprises at least one hypertension score and at least one functionality score. In other embodiments, the set of hypertension metrics may comprise at least one hypertension score, at least one functionality score, and medication use. More than one set of hypertension metrics may be reported and analyzed at a given time. For example, a first set of hypertension metrics recording a pet's current status and a second set of hypertension metrics recording the pet's status at an earlier time may both be analyzed and may also be used to generate one or more recommended actions.

Each hypertension metric may be given equal weight in the analysis, or may also be given greater or less weight than other hypertension metrics included in the analysis. For example, a functionality score may be given greater or less weight with respect to a hypertension score and/or medication use. Whether and/or how to weigh a given hypertension metric may be determined according to the characteristics or needs of a particular pet. As an example, Pet A reports a hypertension score of 8 (on a scale of 1 to 10 where 10 is the most severe hypertension) and a functionality score of 9 (on a scale of 1 to 10 where 10 is highest functioning), while Pet B reports a hypertension score of 8 but a functionality score of 4. The present disclosure provides for the collection, analysis, and reporting of this information, taking into account the differential impact of one hypertension score on a pet's functionality versus that same hypertension score's impact on the functionality of a different pet.

Hypertension metrics may undergo a pre-analysis before inclusion in a set of hypertension metrics and subsequent application of one or more algorithms. For example, a raw score may be converted or scaled according to one or more algorithm(s) developed for a particular pet. In some embodiments, for example, a non-numerical raw score may be converted to a numerical score or otherwise quantified prior to the application of one or more algorithms. Pets and healthcare providers may retain access to raw data (e.g., hypertension metric data prior to any analysis)

Algorithm(s) according, to the present disclosure may analyze the set of hypertension metrics according to any suitable methods known in the art. Analysis may comprise, for example, calculation of statistical averages, pattern recognition, application of mathematical models, factor analysis, correlation, and/or regression analysis. Examples of analyses that may be used herein include, but are not limited to, those disclosed in U.S. Patent Application Publication No. 2012/0246102 A1 the entirety of which is incorporated herein by reference.

The present disclosure further provides for the determination of an aggregated hypertension assessment score. In some embodiments, for example, a set of pairs metrics may be analyzed to generate a comprehensive and/or individualized assessment of hypertension by generating a composite or aggregated score. In such embodiments, the aggregated score may include a combination of at least one hypertension score, at least one functionality score, and medication use. Additional metrics may also be included in the aggregated score. Such metrics may include, but are not limited to, exercise habits, mental well-being, depression, cognitive functioning, medication side effects, etc. Any of the aforementioned types of analyses may be used in determining an aggregated score.

The algorithm(s) may include a software program that may be available for download to an input device in various versions. In some embodiments, for example, the algorithm(s) may be directly downloaded through the Internet or other suitable communications means to provide the capability to troubleshoot a health issue in real-time. The algorithm(s) may also be periodically updated, e.g., provided content changes, and may also be made available for download to an input device.

The methods presently disclosed may provide a healthcare provider with a more complete record of a pet's day-to-day status. By having access to a consistent data stream of hypertension metrics for a pet, a healthcare provider may be able to provide the pet with timely advice and real-time coaching on hypertension management options and solutions. A pet may, for example, seek and/or receive feedback on hypertension management without waiting for an upcoming appointment with a healthcare provider or scheduling a new appointment. Such real-time communication capability may be especially beneficial to provide pets with guidance and treatment options during intervals between appointments with a healthcare provider. Healthcare providers may also be able to monitor a pet's status between appointments to timely initiate, modify, or terminate a treatment plan as necessary. For example, a pet's reported medication use may convey whether the pet is taking too little or too much medication. In some embodiments, an alert may be triggered to notify the pet and/or a healthcare provider of the amount of medication taken, e.g., in comparison to a prescribed treatment plan. The healthcare provider could, for example, contact the pet to discuss the treatment plan. The methods disclosed herein may also provide a healthcare provider with a longitudinal review of how a pet responds to hypertension over time. For example, a healthcare provider may be able to determine whether a given treatment plan adequately addresses a pet's needs based on review of the pet's reported hypertension metrics and analysis thereof according to the present disclosure.

Analysis of pet data according to the methods presently disclosed may generate one or more recommended actions that may be transmitted and displayed on an output device. In some embodiments, the analysis recommends that a pet make no changes to his/her treatment plan or routine. In other embodiments, the analysis generates a recommendation that the pet seek further consultation with a healthcare provider and/or establish compliance with a prescribed treatment plan. In other embodiments, the analysis may encourage a pet to seek immediate medical attention. For example, the analysis may generate an alert to be transmitted to one or more output devices, e.g., a first output device belonging to the pet and a second output device belonging to a healthcare provider, indicating that the pet is in need of immediate medical treatment. In some embodiments, the analysis may not generate a recommended action. Other recommended actions consistent with the present disclosure may be contemplated and suitable according to the treatment plans, needs, and/or preferences for a given pet.

The present disclosure further provides a means for monitoring a pet's medication use to determine when his/her prescription will run out and require a refill. For example, a pet profile may be created that indicates a prescribed dosage and frequency of administration, as well as total number of dosages provided in a single prescription. As the pet reports medication use, those hypertension metrics may be transmitted to a server and stored in a database in connection with the pet profile. The pet profile stored on the database may thus continually update with each added metric and generate a notification to indicate when the prescription will run out based on the reported medication use. The notification may be transmitted and displayed on one or more output devices, e.g., to a pet and/or one or more healthcare providers. In some embodiments, the one or more healthcare providers may include a pharmacist. For example, a pharmacist may receive notification of the anticipated date a prescription will run out in order to ensure that the prescription may be timely refilled.

Pet data can be input for analysis according to the systems disclosed herein through any data-enabled device including, but not limited to, portable/mobile and stationary communication devices, and portable/mobile and stationary computing devices. Non-limiting examples of input devices suitable for the systems disclosed herein include smart phones, cell phones, laptop computers, netbooks, personal computers (PCs), tablet PCs, fax machines, personal digital assistants, and/or personal medical devices. The user interface of the input device may be web-based, such as a web page, or may also be a stand-alone application. Input devices may provide access to software applications via mobile and wireless platforms, and may also include web-based applications.

The input device may receive data by having a user, including, but not limited to, a pet, family member, friend, guardian, representative, healthcare provider, and/or caregiver, enter particular information via a user interface, such as by typing and/or speaking. In some embodiments, a server may send a request for particular information to be entered by the user via an input device. For example, an input device may prompt a user to enter sequentially a set of hypertension metrics, e.g., a hypertension score, a functionality score, and information regarding use of one or more medications (e.g., type of medication, dosage taken, time of day, route of administration, etc.). In other embodiments, the user may enter data into the input device without first receiving a prompt. For example, the user may initiate an application or web-based software program and select an option to enter one or more hypertension metrics. In some embodiments, one or more hypertension scales and/or functionality scales may be preselected by the application or software program. For example, a user may have the option of selecting the type of hypertension scale and/or functionality scale for reporting hypertension metrics within the application or software program. In other embodiments, an application or software program may not include preselected hypertension scales or functionality scales such that a user can employ any hypertension scale and/or functionality scale of choice.

The user interface of an input device may allow a user to associate hypertension metrics with a particular date and/or time of day. For example, a user may report one or more hypertension metrics to reflect a pet's present status. A user may also report one or more hypertension metrics to reflect a pet's status at an earlier time.

Pet data may be electronically transmitted from an input device over a wired or wireless medium to a server, e.g., a remote server. The server may provide access to a database for performing an analysis of the data transmitted, e.g., set of hypertension metrics. The database may comprise auxiliary data for use in the analysis as described above. In some embodiments, the analysis may be automated, and may also be capable of providing real-time feedback to pets and/or healthcare providers.

The analysis may generate one or more recommended actions, and may transmit the recommended action(s) over at wired or wireless medium for display on at least one output device. The at least one output device may include, e.g., portable/mobile and stationary communication devices, and portable/mobile and stationary computing devices. Non-limiting examples of output devices suitable for the systems disclosed herein include smart phones, cell phones, laptop computers, netbooks, personal computers (PCs), tablet PCs, fax machines, personal digital assistants, and/or personal medical devices. In some embodiments, the input device is the at least one output device. In other embodiments, the input device is one of multiple output devices. In some embodiments of the present disclosure, the one or more recommended actions are transmitted and displayed on each of two output devices. In such an example, one output device may belong to a pet and the other device may belong to a healthcare provider.

The present disclosure also contemplates methods and systems in a language suitable for communicating with the pet and/or healthcare provider, including languages other than English.

A pet's medical data may be subject to confidentiality regulations and protection. Transmitting, analyzing, and/or storing information according to the methods and systems disclosed herein may be accomplished through secure means, including HIPPA-compliant procedures and use of password-protected devices, servers, and databases.

The systems and methods presently disclosed may be especially beneficial in outpet, home, and/or on-the-go settings. The systems and methods disclosed herein may also be used as an inpet tool and/or in controlled medication administration such as developing a personalized treatment plan.

In addition to monitoring health parameters, the system can include interventional devices such as a defibrillator. The defibrillator function is enabled by providing electrical energy of a selected energy/power level/voltage/current level or intensity delivered for a selected duration upon sensing certain patterns of undesirable heart activity wherein said undesirable heart activity necessitates an external delivery of a controlled electrical energy pulse for stimulating a selected heart activity. The defibrillator function is enabled by an intelligent defibrillator appliance that operates in a manner similar to the functions of an intelligent ECG appliance with the additional capability of providing external electrical stimuli via for example a wireless contact system pasted on various locations of the torso. The electrical stimuli are delivered in conjunction with the intelligent defibrillator device or the mobile device performing the additional functions of an intelligent defibrillator appliance. The control actions for providing real time stimuli to the heart of electrical pulses, is enabled by the intelligent defibrillator appliance by itself or in conjunction with an external server/intelligent appliance where the protocols appropriate for the specific individual are resident. The defibrillation actions are controlled in conjunction with the real time ECG data for providing a comprehensive real-time solution to the individual suffering from abnormal or life-threatening heart activity/myocardial infraction. Additionally, by continuously wearing the paste on wireless contacts that can provide the electrical impulse needed, the individual is instantaneously able to get real time attention/action using a specifically designed wearable intelligent defibrillator appliance or a combination of an intelligent ECG plus defibrillator appliance. Further the mobile device such as a cellular telephone or other wearable mobile devices can be configured with the appropriate power sources and the software for performing the additional functions of an intelligent defibrillator appliance specifically tailored to the individual.

The cellular telephone/mobile device can receive signals from the ECG machine/appliance or as an intermediary device that transmits/receives the ECG data and results from a stationary or portable ECG appliance. The ability of the individual to obtain an ECG profile of the heart at a selected time and in a selected location is critical to getting timely attention and for survival. Getting attention within 10 to 20 minutes of a heart attack is crucial beyond that the chances for survival diminish significantly. The smart phone helps the pet to quickly communicate his/her location and or discover the location of the nearest health care facility that has the requisite cardiac care facilities and other facilities. The mobile device that the individual is carrying on the person is enabled to provide the exact location of the individual in conjunction with the global positioning system. In addition, the system is enabled to provide the directions and estimated travel time to/from the health care facility to the specific mobile device/individual.

Yet other intervention can include music, image, or video. The music can be synchronized with respect to a blood pulse rate in one embodiment, and in other embodiments to biorhythmic signal—either to match the biorhythmic signal, or, if the signal is too fast or too slow, to go slightly slower or faster than the signal, respectively. In order to entrain the user's breathing, a basic melody is preferably played which can be easily identified by almost all users as corresponding to a particular phase of respiration. On top of the basic melody, additional layers are typically added to make the music more interesting, to the extent required by the current breathing rate, as described hereinabove. Typically, the basic melody corresponding to this breathing includes musical cords, played continuously by the appropriate instrument during each phase. For some applications, it is desirable to elongate slightly the length of one of the respiratory phases, typically, the expiration phase. For example, to achieve respiration which is 70% expiration and 30% inspiration, a musical composition written for an E:I ratio of 2:1 may be played, but the expiration phase is extended by a substantially-unnoticed 16%, so as to produce the desired respiration timing. The expiration phase is typically extended either by slowing down the tempo of the notes therein, or by extending the durations of some or all of the notes.

Although music for entraining breathing is described hereinabove as including two phases, it will be appreciated by persons skilled in the art that the music may similarly include other numbers of phases, as appropriate. For example, user may be guided towards breathing according to a 1:2:1:3 pattern, corresponding to inspiration, breath holding (widely used in Yoga), expiration, and post-expiratory pause (rest state).

In one embodiment, the volume of one or more of the layers is modulated responsive to a respiration characteristic (e.g., inhalation depth, or force), so as to direct the user to change the characteristic, or simply to enhance the user's connection to the music by reflecting therein the respiration characteristic. Alternatively, or additionally, parameters of the sound by each of the musical instruments may be varied to increase the user's enjoyment. For example, during slow breathing, people tend to prefer to hear sound patterns that have smoother structures than during fast breathing and/or aerobic exercise.

Further alternatively or additionally, random musical patterns and/or digitized natural sounds (e.g., sounds of the ocean, rain, or wind) are added as a decoration layer, especially for applications which direct the user into very slow breathing patterns. The inventor has found that during very slow breathing, it is desirable to remove the user's focus from temporal structures, particularly during expiration.

Still further alternatively or additionally, the server maintains a musical library, to enable the user to download appropriate music and/or music-generating patterns from the Internet into device. Often, as a user's health improves, the music protocols which were initially stored in the device are no longer optimal, so the user downloads the new protocols, by means of which music is generated that is more suitable for his new breathing training. The following can be done:

obtaining clinical data from one or more laboratory test equipment and checking the data on a blockchain;

obtaining genetic clinical data from one or more genomic equipment and storing genetic markers in the EMR/HER including germ line data and somatic data over time;

obtaining clinical data from a primary care or a specialist physician database;

obtaining clinical data from an in-pet care database or from an emergency room database;

saving the clinical data into a clinical data repository;

obtaining health data from fitness devices or from mobile phones;

obtaining behavioral data from social network communications and mobile device usage patterns;

saving the health data and behavioral data into a health data repository separate from the clinical data repository; and

providing a decision support system (DSS) to apply genetic clinical data to the subject, and in case of an adverse event for a drug or treatment, generating a drug safety signal to alert a doctor or a manufacturer, wherein the DSS includes rule-based alerts on pharmacogenetics, oncology drug regimens, wherein the DSS performs ongoing monitoring of actionable genetic variants.

FIG. 3F illustrates one embodiment of a system for collaboratively treating a pet with a disease such as cancer. In this embodiment, a treating physician/doctor logs into a consultation system 1 and initiates the process by clicking on “Create New Case” (500). Next, the system presents the doctor with a “New Case Wizard” which provides a simple, guided set of steps to allow the doctor to fill out an “Initial Assessment” form (501). The doctor may enter Pet or Subject Information (502), enter Initial Assessment of pet/case (504), upload Test Results, Subject Photographs and X-Rays (506), accept Payment and Service Terms and Conditions (508), review Summary of Case (510), or submit Forms to a Al machine based “consultant” such as a Hearing Service Al Provider (512). Other clinical information for the cancer subject includes the imaging or medical procedure directed towards the specific disease that one of ordinary skill in the art can readily identify. The list of appropriate sources of clinical information for cancer includes but it is not limited to: CT scan, MRI scan, ultrasound scan, bone scan, PET Scan, bone marrow test, barium X-ray, endoscopy, lymphangiogram, IVU (Intravenous urogram) or IVP (IV pyelogram), lumbar puncture, cystoscopy, immunological tests (anti-malignant antibody screen), and cancer marker tests.

After the case has been submitted, the Al Machine Consultant can log into the system 1 and consult/process the case (520). Using the Treating Doctors Initial Assessment and Photos/X-Rays, the Consultant will click on “Case Consultation” to initiate the “Case Consultation Wizard” (522). The consultant can fill out the “Consultant Record Analysis” form (524). The consultant can also complete the “Prescription Form” (526) and submit completed forms to the original Treating Doctor (528). Once the case forms have been completed by the Consulting Doctor, the Treating Doctor can access the completed forms using the system. The Treating Doctor can either accept the consultation results (i.e. a pre-filled Prescription form) or use an integrated messaging system to communicate with the Consultant (530). The Treating Doctor can log into the system (532), click on Pet Name to review (534), review the Consultation Results (Summary Letter and pre-filled Prescription Form) (536). If satisfied, the Treating Doctor can click “Approve Treatment” (538), and this will mark the case as having being approved (540). The Treating Doctor will be able to print a copy of the Prescription Form and the Summary Letter for submission to hearing aid manufacturer or provider (542). Alternatively, if not satisfied, the Treating Doctor can initiate a computer dialog with the Consultant by clicking “Send a Message” (544). The Treating Doctor will be presented with the “Send a Message” screen where a message about the case under consultation can be written (546). After writing a message, the Treating Doctor would click “Submit” to send the message to the appropriate Consultant (548). The Consultant will then be able to reply to the Treating Doctor's Message and send a message/reply back to the Treating Doctor (550).

Blockchain Authentication

Since the collar/chest/foot/ITE sensors are loT machines, the device can negotiate contracts on their own (without human) and exchange items of value by presenting an open transaction on the associated funds in their respective wallets. Blockchain token ownership is immediately transferred to a new owner after authentication and verification, which are based on network ledgers within a peer-to-peer network, guaranteeing nearly instantaneous execution and settlement.

A similar process is used to provide secure communications between IoT devices, which is useful for edge loT devices. The industrial world is adding billions of new IoT devices and collectively these devices generate many petabytes of data each day. Sending all of this data to the cloud is not only very cost prohibitive but it also creates a greater security risk. Operating at the edge ensures much faster response times, reduced risks, and lower overall costs. Maintaining close proximity to the edge devices rather than sending all data to a distant centralized cloud, minimizes latency allowing for maximum performance, faster response times, and more effective maintenance and operational strategies. In addition to being highly secure, the system also significantly reduces overall bandwidth requirements and the cost of managing widely distributed networks.

In some embodiments, the described technology provides a peer-to-peer cryptographic currency trading method for initiating a market exchange of one or more Blockchain tokens in a virtual wallet for purchasing an asset (e.g., a security) at a purchase price. The system can determine, via a two-phase commit, whether the virtual wallet has a sufficient quantity of Blockchain tokens to purchase virtual assets (such as electricity only from renewable solar/wind/ . . . sources, weather data or location data) and physical asset (such as gasoline for automated vehicles) at the purchase price. In various embodiments, in response to verifying via the two-phase commit that the virtual wallet has a sufficient quantity of Blockchain tokens, the loT machine purchases (or initiates a process in furtherance of purchasing) the asset with at least one of the Blockchain tokens. In one or more embodiments, if the described technology determines that the virtual wallet has insufficient Blockchain tokens for purchasing the asset, the purchase is terminated without exchanging Blockchain tokens.

The present system provides smart contract management with modules that automates the entire lifecycle of a legally enforceable smart contract by providing tools to author the contract so that it is both judge/arbitrator/lawyer readable and machine readable, and ensuring that all contractual obligations are met by integrating with appropriate execution systems, including traditional court system, arbitration system, or on-line enforcement system. Different from the blockchain/bitcoin contract system where payment is made in advance and released when the conditions are electronically determined to be satisfied, this embodiment creates smart contracts that are verifiable, trustworthy, yet does not require advance payments that restrict the applicability of smart contracts. The system has a contract management system (CMS) that helps users in creating smart contracts for deployment. After template creation, in one embodiment, the functionality of the flow diagram of FIG. 13A is implemented by software stored in memory and executed by a processor. In other embodiments, the functionality can be performed by hardware, or any combination of hardware and software.

In implementation, the blockchain is decentralized and does not require a central authority for creation, processing or verification and comprises a public digital ledger of all transactions that have ever been executed on the blockchain and wherein new blocks are added to the blockchain in a linear, chronological order. The public digital ledger of the blockchain comprises transactions and blocks. Blocks in the blockchain record and confirm when and in what sequence transactions are entered and logged into the blockchain. The transactions comprise desired electronic content stored in the blockchain. The desired electronic content includes a financial transaction. The financial transaction includes a cryptocurrency transaction, wherein the cryptocurrency transaction includes a BITCOIN or an ETHEREUM transaction. An identifier for the received one or more blocks in the blockchain includes a private encryption key.

Medical History

The above permissioned blockchain can be used to share sensitive medical data with different authorized institutions. The institutions are trusted parties and vouched for by the trusted point. A Pet-Provider Relationship (PPR) Smart Contract is issued when one node from a trusted institution stores and manages medical records for the pet. The PPR defines an assortment of data pointers and associated access permissions that identify the records held by the care provider. Each pointer consists of a query string that, when executed on the provider's database, returns a subset of pet data. The query string is affixed with the hash of this data subset, to guarantee that data have not been altered at the source. Additional information indicates where the provider's database can be accessed in the network, i.e. hostname and port in a standard network topology. The data queries and their associated information are crafted by the care provider and modified when new records are added. To enable pets to share records with others, a dictionary implementation (hash table) maps viewers' addresses to a list of additional query strings. Each string can specify a portion of the pet's data to which the third party viewer is allowed access. For SQL data queries, a provider references the pet's data with a SELECT query on the pet's address. For pets uses an interface that allows them to check off fields they wish to share through a graphical interface. The system formulates the appropriate SQL queries and uploads them to the PPR on the blockchain.

In another embodiment, the system includes two look up tables, a global registration look up table (GRLT) where all participants (medical institutions and pets) are recorded with name or identity string, blockchain address for the smart contract, and Pet-Provider lookup table (PPLT). This is maintained by a trusted host authority such as a government health authority or a government payor authority. One embodiment maps participant identification strings to their blockchain address or Ethereum address identity (equivalent to a public key). Terms in the smart contract can regulate registering new identities or changing the mapping of existing ones. Identity registration can thus be restricted only to certified institutions. The PPLT maps identity strings to an address on the blockchain.

Pets can poll their PPLT and be notified whenever a new relationship is suggested or an update is available. Pets can accept, reject or delete relationships, deciding which records in their history they acknowledge. The accepting or rejecting relationships is done only by the pets. To avoid notification spamming from malicious participants, only trusted providers can update the status variable. Other contract terms or rules can specify additional verifications to confirm proper actor behavior.

When Provider 1 adds a record for a new pet, using the GRLT on the blockchain, the pet's identifying information is first resolved to their matching Ethereum address and the corresponding PPLT is located. Provider 1 uses a cached GRLT table to look up any existing records of the pet in the PPLT. For all matching PPLTs, Provider 1 broadcasts a smart contract requesting pet information to all matching PPLT entries. If the cache did not produce a result for the pet identity string or blockchain address, Provider 1 can send a broadcast requesting institutions who handles the pet identity string or the blockchain address to all providers. Eventually, Provider 2 responds with its addresses. Provider 2 may insert an entry for Provider 1 into its address resolution table for future use. Provider 1 caches the response information in its table and can now pull information from Provider 2 and/or supplement the information known to Provider 2 with hashed addresses to storage areas controlled by Provider 1.

Next, the provider uploads a new PPR to the blockchain, indicating their stewardship of the data owned by the pet's Ethereum address. The provider node then crafts a query to reference this data and updates the PPR accordingly. Finally, the node sends a transaction which links the new PPR to the pet's PPLT, allowing the pet node to later locate it on the blockchain.

A Database Gatekeeper provides an off-chain, access interface to the trusted provider node's local database, governed by permissions stored on the blockchain. The Gatekeeper runs a server listening to query requests from clients on the network. A request contains a query string, as well as a reference to the blockchain PPR that warrants permissions to run it. The request is cryptographically signed by the issuer, allowing the gatekeeper to confirm identities. Once the issuer's signature is certified, the gatekeeper checks the blockchain contracts to verify if the address issuing the request is allowed access to the query. If the address checks out, it runs the query on the node's local database and returns the result over to the client.

A pet owner selects data to share and updates the corresponding PPR with the third-party address and query string. If necessary, the pet's node can resolve the third party address using the GRLT on the blockchain. Then, the pet node links their existing PPR with the care provider to the third-party's Summary Contract. The third party is automatically notified of new permissions, and can follow the link to discover all information needed for retrieval. The provider's Database Gatekeeper will permit access to such a request, corroborating that it was issued by the pet on the PPR they share.

In one embodiment that handles persons without previous blockchain history, admitting procedures are performed where the person's personal data is recorded and entered into the blockchain system. This data may include: name, address, home and work telephone number, date of birth, place of employment, occupation, emergency contact information, insurance coverage, reason for hospitalization, allergies to medications or foods, and religious preference, including whether or not one wishes a clergy member to visit, among others. Additional information may include past hospitalizations and surgeries, advance directives such as a living will and a durable power to attorney. During the time spent in admitting, a plastic bracelet will be placed on the person's wrist with their name, age, date of birth, room number, and blockchain medical record reference on it.

The above system can be used to connect the blockchain with different EHR systems at each point of care setting. Any time a pet is registered into a point of care setting, the EHR system sends a message to the GRLT to identify the pet if possible. In our example, Pet A is in registration at a particular hospital. The PPLT is used to identify Pet A as belonging to a particular plan. The smart contracts in the blockchain automatically updates Pet A's care plan. The blockchain adds a recommendation to put Pet A by looking at the complete history of treatments by all providers and optimizes treat. For example, the system can recommend the pet be enrolled in a weight loss program after noticing that the pet was treated for sedentary lifestyle, had history of hypertension, and the pet family history indicates a potential heart problem. The blockchain data can be used for predictive analytics, allowing pets to learn from their family histories, past care and conditions to better prepare for healthcare needs in the future. Machine learning and data analysis layers can be added to repositories of healthcare data to enable a true “learning health system” can support an additional analytics layer for disease surveillance and epidemiological monitoring, physician alerts if pets repeatedly fill and abuse prescription access.

In one embodiment, an IOT medical device captures pet data in the hospital and automatically communicates data to a hospital database that can be shared with other institutions or doctors. First, the pet ID and blockchain address is retrieved from the pet's wallet and the medical device attaches the blockchain address in a field, along with other fields receiving pet data. Pet data is then stored in a hospital database marked with the blockchain address and annotated by a medical professional with interpretative notes. The notes are affiliated with the medical professional's blockchain address and the PPR block chain address. A professional can also set up the contract terms defining a workflow. For example, if the device is a blood pressure device, the smart contract can have terms that specify dietary restrictions if the pet is diabetic and the blood pressure is borderline and food dispensing machines only show items with low salt and low calorie, for example.

The transaction data may consist of a Colored Coin implementation (described in more detail at https://en.bitcoin.it/wiki/Colored_Coins which is incorporated herein by reference), based on Open Assets (described in more detail at https://github.com/OpenAssets/open-assets-protocol/blob/master/specification.mediawiki which is incorporated herein by reference), using on the OP_RETURN operator. Metadata is linked from the Blockchain and stored on the web, dereferenced by resource identifiers and distributed on public torrent files. The colored coin specification provides a method for decentralized management of digital assets and smart contracts (described in more detail at https://github.com/ethereum/wiki/wiki/White-Paper which is incorporated herein by reference.) For our purposes the smart contract is defined as an event-driven computer program, with state, that runs on a blockchain and can manipulate assets on the blockchain. So a smart contract is implemented in the blockchain scripting language in order to enforce (validate inputs) the terms (script code) of the contract.

Pet Behavior and Risk Pool Rated Health Plans

With the advent of personal health trackers, new health plans are rewarding consumers for taking an active part in their wellness. The system facilitates open distribution of the consumers wellness data and protect it as PHR must be, and therefore prevent lock-in of consumers, providers and payers to a particular device technology or health plan. In particular, since PHR data is managed on the blockchain a consumer and/or company can grant access to a payer to this data such that the payer can perform group analysis of an individual or an entire company's employee base including individual wellness data and generate a risk score of the individual and/or organization. Having this information, payers can then bid on insurance plans tailored for the specific organization. Enrollment then, also being managed on the blockchain, can become a real-time arbitrage process. The pseudo code for the smart contract to :implement a pet behavior based health plan is as follows:
store mobile fitness data
store pet data in keys with phr_info, claim_info, enrollment_info
for each pet:
   add up all calculated risk for the pet
   determine risk score based on mobile fitness data
   update health plan cost based on pet behavior

Pet and Provider Data Sharing

A pet's Health BlockChain wallet stores all assets, which in turn store reference ids to the actual data, whether clinical documents in HL7 or FHIR format, wellness metrics of activity and sleep patterns, or claims and enrollment information. These assets and control of grants of access to them is afforded to the pet alone. A participating provider can be given full or partial access to the data instantaneously and automatically via enforceable restrictions on smart contracts.

Utilizing the Health BlockChain the access to a pet's PHR can be granted as part of scheduling an appointment, during a referral transaction or upon arrival for the visit. And, access can just as easily be removed, all under control of the pet.

Upon arrival at the doctor's office, an application automatically logs into a trusted provider's wireless network. The app is configured to automatically notify the provider's office of arrival and grant access to the pet's PHR. At this point the attending physician will have access to the pet's entire health history. The pseudo code for the smart contract to implement a pet and provider data sharing is as follows.
Pet Owner downloads apps and provide login credential and logs into the provider wireless network
Pet owner verifies that the provider wireless network belongs to a pet trusted provider list
Upon entering provider premise, system automatically logs in and grants access to provider
Pet check in data is automatically communicated with provider system to provide PHR
Provider system synchronizes files and obtain new updates to the pet PHR and flags changes to provider.

Pet Data Sharing

Pet's PHR data is valuable information for their personal health profile in order to provide Providers the necessary information for optimal health care delivery. In addition this clinical data is also valuable in an aggregate scenario of clinical studies where this information is analyzed for diagnosis, treatment and outcome. Currently this information is difficult to obtain due to the siloed storage of the information and the difficulty on obtaining pet permissions.

Given a pet Health BlockChain wallet that stores all assets as reference ids to the actual data. These assets can be included in an automated smart contract for clinical study participation or any other data sharing agreement allowed by the pet. The assets can be shared as an instance share by adding to the document a randomized identifier or nonce, similar to a one-time use watermark or serial number, a unique asset (derived from the original source) is then generated for a particular access request and included in a smart contract as an input for a particular request for the pet's health record information. A pet can specify their acceptable terms to the smart contract regarding payment for access to PHR, timeframes for acceptable access, type of PHR data to share, length of history willing to be shared, de-identification thresholds or preferences, specific attributes of the consumer of the data regarding trusted attributes such as reputation, affiliation, purpose, or any other constraints required by the pet. Attributes of the pet's data are also advertised and summarized as properties of the smart contract regarding the type of diagnosis and treatments available. Once the pet has advertised their willingness to share data under certain conditions specified by the smart contract it can automatically be satisfied by any consumer satisfying the terms of the pet and their relevance to the type of PHR needed resulting in a automated, efficient and distributed means for clinical studies to consume relevant PHR for analysis. This process provides an automated execution over the Health BlockChain for any desired time period that will terminate at an acceptable statistical outcome of the required attained significance level or financial limit. The pseudo code for the smart contract to implement automated pet data sharing is as follows.
Pet Owner download apps and provide login credential and logs into the clinical trial provider wireless network
Pet owner verifies that the provider wireless network belongs to a pet trusted provider list
Upon entering provider premise, system automatically logs in and grants access to provider
Pet check in data is automatically communicated with provider system to provide clinical trial data

In one embodiment, a blockchain entry is added for each touchpoint of the medication as it goes through the supply chain from manufacturing where the prescription package serialized numerical identification (SNI) is sent to wholesalers who scan and record the SNI and location and then to distributors, repackagers, and pharmacies, where the SNI/location data is recorded at each touchpoint and put on the blockchain. The medication can be scanned individually, or alternatively can be scanned in bulk. Further, for bulk shipments with temperature and shock sensors for the bulk package, temperature/shock data is captured with the shipment or storage of the medication.

A smart contract assesses against product supply chain rule and can cause automated acceptance or rejection as the medication goes through each supply chain touchpoint. The process includes identifying a prescription drugs by query of a database system authorized to track and trace prescription drugs or similar means for the purpose of monitoring the movements and sale of pharmaceutical products through a supply chain; a.k.a. e-pedigree trail; serialized numerical identification (SNI), stock keeping units (SKU), point of sale system (POS), systems etc. in order to compare the information; e.g. drug name, manufacturer, etc. to the drug identified by the track and trace system and to ensure that it is the same drug and manufacturer of origin. The process can verify authenticity and check pedigree which can be conducted at any point along the prescription drug supply chain; e.g. wholesaler, distributor, doctor's office, pharmacy. The most optimal point for execution of this process would be where regulatory authorities view the greatest vulnerability to the supply chain's integrity. For example, this examination process could occur in pharmacy operations prior to containerization and distribution to the pharmacy for dispensing to pets.

An authenticated prescription drug with verified drug pedigree trail can be used to render an informational object, which for the purpose of illustration will be represented but not be limited to a unique mark; e.g. QR Code, Barcode, Watermark, Stealth Dots, Seal or 2 Dimensional graphical symbol, hereinafter called a certificate, seal, or mark. An exemplary embodiment for use of said certificate, mark, or seal can be used by authorized entities as a warrant of the prescription drug's authenticity and pedigree. For example, when this seal is appended to a prescription vial presented to a pet by a licensed pharmacy, it would represent the prescription drug has gone through an authentication and logistics validation process authorized by a regulatory agency (s); e.g. HHS, FDA, NABP, VIPP, etc. An exemplary embodiment for use of said certificate, mark or seal would be analogous to that of the functioning features, marks, seals, and distinguishing characteristics that currently authenticate paper money and further make it difficult to counterfeit. Furthermore, authorized agents utilizing the certificate process would be analogous to banks participating in the FDIC program.

A user; e.g. pet equipped with the appropriate application on a portable or handheld device can scan the certificate, mark or seal and receive an audible and visible confirmation of the prescription drug's name, and manufacturer. This will constitute a confirmation of the authenticity of the dispensed prescription drug. Extensible use of the certificate, mark, or seal will include but not be limited to; gaining access to website (s) where additional information or interactive functions can be performed; e.g. audible narration of the drug's characteristics and physical property descriptions, dosing, information, and publications, etc. A user; e.g. pet equipped with the appropriate application on a portable or handheld device can scan the certificate, mark, or seal and be provided with notifications regarding; e.g. immediate recall of the medication, adverse events, new formulations, critical warnings of an immediate and emergency nature made by prescription drug regulatory authorities and, or their agents. A user; e.g. pet equipped with a portable or handheld device with the appropriate application software can use the portable and, or handheld device to store prescription drug information in a secure, non-editable format on their device for personal use; e.g. MD's Office Visits, Records Management, Future Authentications, Emergency use by first responders etc. A user; e.g. pet equipped with the appropriate application on a portable or handheld device can scan the drug via an optical scan, picture capture, spectroscopy or other means of identifying its physical properties and characteristics; e.g. spectral signature, size, shape, color, texture, opacity, etc and use this data to identify the prescription drug's name, and manufacturer. A user; e.g. pet equipped with the appropriate application on a portable or handheld device and having the certification system can receive updated information (as a subscriber in a client/server relationship) on a continuing or as needed ad hoc basis (as permitted) about notifications made by prescription drug regulatory authorities regarding; e.g. immediate recall of medications, adverse events, new formulations and critical warnings of an immediate and emergency nature. A user; e.g. pet, subscriber to the certificate system equipped with the appropriate application on a portable or handheld device will be notified by audible and visible warnings of potential adverse affects between drug combinations stored in their device's memory of previously “Certified Drugs.” A user; e.g. pet subscriber to the certification system equipped with the appropriate application on a portable or handheld device will receive notification of potential adverse affects from drug combinations, as reported and published by medical professionals in documents and databases reported to; e.g. Drug Enforcement Administration (DEA), Health and Human Services, (HHS) Food and Drug Administration, (FDA) National Library of Medicines, (NLM) and their agents; e.g., Daily Med, Pillbox, RX Scan, PDR, etc.

1. A method for prescription drug authentication by receiving a certificate representing manufacturing origin and distribution touchpoints of a prescription drug on a blockchain.

2. A method of claim 1, comprising retrieving active pharmaceutical ingredients (API) and inactive pharmaceutical ingredients (IPI) from the blockchain.

3. A method of claim 2, comprising authenticating the drug after comparing the API and IPI with data from Drug Enforcement Administration (DEA) Health and Human Services, (HHS) Food and Drug Administration, (FDA) National Library of Medicines, (NLM) etc. for the purpose of identifying the prescription drug'(s) and manufacture name indicated by those ingredients.

4. A method of claim 1, comprising tracing the drug through a supply chain from manufacturer to retailer, dispenser with Pedigree Trail, Serialized Numerical Identification (SNI), Stock Keeping Units (SKU), Point of Sale System (POS) E-Pedigree Systems.

5. A method of claim 1, comprising generating a certificate, seal, mark and computer scannable symbol such as 2 or 3 dimensional symbol; e.g. QR Code, Bar Code, Watermark, Stealth Dots, etc.

Recognition of Activity Pattern and Tracking of Calorie Consumption

The learning system can be used to detect and monitor user activities as detected by the collar, chest, foot, or ITE sensors. For example, the accelerometer senses vibration—particularly the vibration of a vehicle such as a ski or mountain bike—moving along a surface, e.g., a ski slope or mountain bike trail. This voltage output provides an acceleration spectrum over time; and information about airtime can be ascertained by performing calculations on that spectrum. Based on the information, the system can reconstruct the movement path, the height, the speed, among others and such movement data is used to identify the exercise pattern. For example, the user may be interested in practicing mogul runs, and the system can identify foot movement and speed and height information of the pet and present the information post exercises as feedback. Alternatively, the system can make live recommendations to improve performance to the pet owner.

In one implementation a Hidden Markov Model (HMM) is used to track pet motor skills or sport movement patterns. The sequence of pet motions can be classified into several groups of similar postures and represented by mathematical models called model-states. A model-state contains the extracted features of body signatures and other associated characteristics of body signatures. Moreover, a gait or posture graph is used to depict the inter-relationships among all the model-states, defined as PG(ND,LK), where ND is a finite set of nodes and LK is a set of directional connections between every two nodes. The directional connection links are called posture links. Each node represents one model-state, and each link indicates a transition between two model-states. In the posture graph, each node may have posture links pointing to itself or the other nodes.

In the pre-processing phase, the system obtains the pet body profile and the body signatures to produce feature vectors. In the model construction phase, the system generates a posture graph, examine features from body signatures to construct the model parameters of HMM, and analyze pet body contours to generate the model parameters of ASMs. In the motion analysis phase, the system uses features extracted from the body signature sequence and then applies the pre-trained HMM to find the posture transition path, which can be used to recognize the motion type. Then, a motion characteristic curve generation procedure computes the motion parameters and produces the motion characteristic curves. These motion parameters and curves are stored over time, and if differences for the motion parameters and curves over time is detected, the system then runs the sport enthusiast through additional tests to confirm the detected motion.

In one embodiment, exercise motion data acquired by the accelerometer or multi-axis force sensor is analyzed, as will be discussed below, in order to determine the motion of each leg or wing stroke during the session (i.e., horizontal vertical or circular). In another embodiment, data obtained from the gyroscope, if one is used, typically does not require a complex analysis. To determine which side of the body is exercise, the gyroscope data is scanned to determine when the rotational orientation is greater than 180 degrees, indicating the left side, and when it is less than 180 degrees, indicating the right side. As explained above, top and bottom and gum brushing information can also be obtained, without any calculations, simply by examining the data. The time sequence of data that is acquired during exercise and analyzed as discussed above can be used in a wide variety of ways.

The sensors can be used to detect normal/abnormal activities of life for the pet. Animals behave in ways to enhance their lifetime fitness, choosing from their behavioral repertoire according to their environmental and internal circumstance. Their movements and/or postural patterns, both of which can be quantified using accelerometers. For example, orthogonally-orientated tri-axial accelerometers can provide high resolution (infra-second) data to define tag-orientation with respect to gravity (if no other forces are operating), and therefore animal posture (the ‘static’ acceleration component), as well as the extent of movement given by the dynamic component of acceleration. In addition to accelerometer, tri-axial sensors measuring angular rotation, notably gyroscopes or magnetometers, both can be combined with accelerometers in inertial measurement units. Machine learning is applied to these sensor outputs to identify the animal activity, be it resting, sleeping, running, flying, among others. Features in the data can be extracted using fast fourier transformation on metrics prior to learning/training. The system can isolate single m-print trajectories for a repetitive or periodic behavior. The features can be isolated using a Fast Fourier Transformation (FFT). The FFT can be applied to identify the frequencies of limb movement from acceleration data and periodic patterns in depth profiles such as those associated with diel vertical migration. Periodicity in behavior measured with the TriMag should be readily identifiable with signal processing tools such as the FFT due to the low signal-to-noise ratio to allow periodic behaviors to be isolated, even where they do not result in change in either acceleration or environmental parameters (e.g. dive depth in dolphin).

The learning machine can identify gait, or the pattern of footsteps at different speeds. Each gait is distinguished by a specific pattern of footfall and rhythm. For example, dogs have four main gaits. From slowest to fastest, they are the walk, trot, canter and gallop. Between the walk and trot is a transitional gate called the amble.

When a dog walks, it first moves one rear leg forward, then the front foot on that same side. Then it moves the other rear foot forward, then the front foot on that side. The pattern of footfall for the walk is right rear, right front, left rear, left front (this is repeated). As a dog walks, sometimes two feet are on the ground; other times there are three. The walk is the only dog gait in which three feet can be on the ground at the same time. If monitoring devices see three of the dog's feet on the ground, monitoring devices know the dog is walking.

As a walking dog speeds up, each rear foot that steps forward is quickly followed by the front foot on the same side. Eventually, it begins to look as if the two feet on the same side of the dog's body are moving forward together.

An amble is essentially a fast walk. An ambling dog looks very ungainly. The rear end sways from side to side and the dog doesn't pick up the rear feet, often scuffling them on the ground. But an ambling dog often moves at the same speed as a dog moving at an easy trot. This wasted energy is why the amble is not a preferred gait and should only be used briefly when transitioning from a walk to a trot, or when a tired dog wants to rest the muscles used for trotting, and use its legs in a different way for a while.

If an ambling dog gradually speeds up, the two feet on the same side of the body that are moving forward together end up bearing all of the dog's weight. Then the two legs on the other side of the body move forward and bear the dog's weight. Now the dog is pacing. In a pace, only two feet are on the ground at any given time, either both right feet or both left feet. Sometimes, owners inadvertently train their dogs to pace by cuing the dog to gradually speed up, thus moving naturally from a walk to an amble to a pace. If this happens frequently, the pace can become the dog's habitual gait. Another reason some dogs pace is because they have more angulation in the rear legs than in the front, causing those angulated rear legs to strike the front legs when the dog is trotting. To avoid this, some dogs will pace, moving the front and rear legs on the same side forward together, thus avoiding interference.

The trot is truly the dog's most efficient gait. When trotting, a dog moves diagonal front and rear feet forward. First, two diagonal front and rear feet move forward (for example, right front-left rear). Then, the dog's entire body is suspended in the air for a moment. Then, the other diagonal front and rear feet move forward (for example, left front-right rear). The trot is the best gait to use when walking a dog for exercise because it's the only gait that requires each side of the dog's body to work equally hard.

The canter is the main gait dogs use in the sport of agility. The pattern of footfall for this gait has two variations. In the classical canter, first one rear foot moves forward, then the other rear foot and the diagonal front foot move forward together, then finally, the last front foot moves. The order of footfall is right rear, left rear-right front, left front, or the reverse of this pattern. The classical canter is how horses canter. But dogs use this form of the canter only about 10 percent of the time. The rest of the time, dogs use the rotary canter. In the rotary canter, the order of footfall would be either right rear, left rear-left front, right front or the reverse of this pattern. The rotary canter allows dogs to turn sharply and with greater drive from the rear.

The gallop starts with the dog's spine flexed and two rear feet on the ground, one foot (the lead foot) slightly ahead of the other. The dog then extends its spine, stretching its front feet forward, which hit the ground with one foot (the lead foot) slightly ahead of the other. The dog then flexes the spine to bring the rear feet forward to start the cycle again. When the dog uses the same lead foot in the front and rear, the gait is called the classical gallop—the same type of gallop used by horses. But when the front legs are on a different lead from the rear—it is a rotary gallop—the preferential gait for dogs.

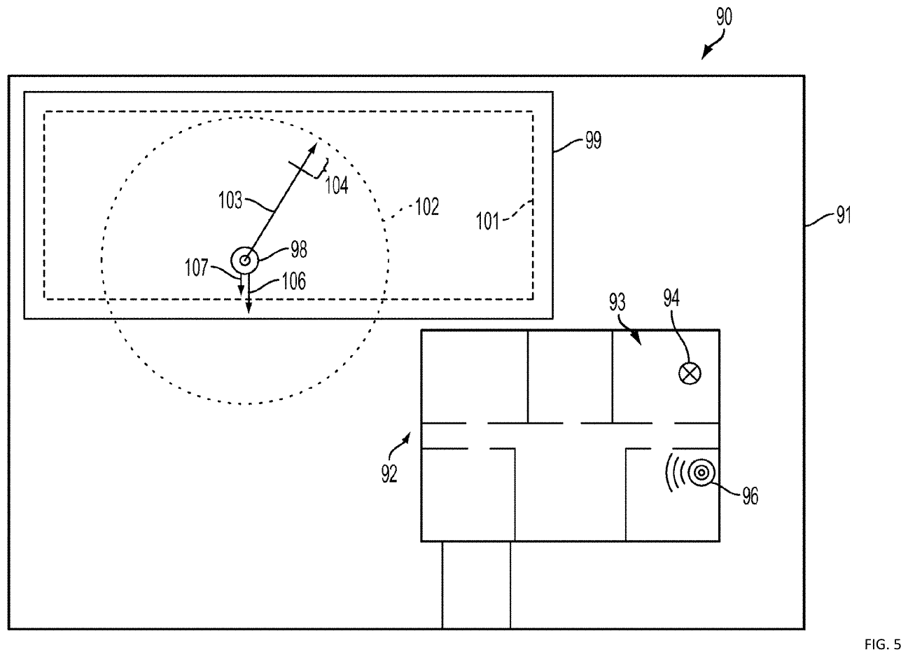

FIG. 4 depicts a side view of the dog's collar 15. An on/off switch 46 is located on the side of the electronics module 19, directly adjacent to an LED 48 that indicates whether the collar's electronic components are on or off. Self-adjusting collar strap 17 attaches to the electronics module 19 via strap retainers 44. Shocking prongs 47 protrude through holes in strap 17 in order to maintain contact with the dog's body. The training collar 15 consists of two major components: an electronics module 19 and a self-adjusting strap 17. The electronic components are housed in a generally waterproof case 26. The electronics module 19 is powered by battery, which is accessible via battery compartment access panel. Electronics module receives power and data via connection ports, which include a USB connector and a power connector. A board serves as the infrastructure for the electronic components contained in the module, including odor sensors, input/output electronics, WiFi/Bluetooth chip, sound synthesizer, GPS chip, cellular transceiver, and microprocessor. Electronics module 19 also contains speaker or suitable acoustic device, which is located directly beneath case perforations in order to produce optimal sound quality at the dog's hearing frequency range. Additional embodiments include electronic components used for monitoring and recording physiological data, such as the dog's pulse rate or body temperature.

In more details, the odor sensor includes a fan module, a gas molecule sensor module, a control unit and an output unit. The fan module is used to pump air actively to the gas molecule sensor module. The gas molecule sensor module detects the air pumped into by the fan module. The gas molecule sensor module at least includes a gas molecule sensor which is covered with a compound. The compound is used to combine preset gas molecules. The control unit controls the fan module to suck air into the electronic nose device. Then the fan module transmits an air current to the gas molecule sensor module to generate a detected data. The output unit calculates the detected data to generate a calculation result and outputs an indicating signal to an operator or compatible host computer according to the calculation result. The gas molecule sensor module detects the incoming air pumped by the fan module. The gas molecule sensor module at least includes a gas molecule sensor covered with a compound for combining preset gas molecules and detects air to generate an electrical signal (such as voltage, current, frequency or phase). In an embodiment, examples of the gas molecule sensor include, without limitation, a piezoelectric quartz crystal, surface acoustic wave material, electrochemistry material, optical fiber, surface plasma resonance and metal oxide semiconductor. In an embodiment, the above mentioned compound for combining at least a preset gas molecule may be ZnO, NiO, Fe2O3, TiO2, CdSnO3, SnO2, WO3 and Au nanoparticle; WO3+SnO2, WO3+ZnO, TiO2+ZnO and WO3+Fe2O3 hybrid nanoparticle; CYS-LYS-ARG-GLN-HIS-PRO-GLY-LYS-ARG-CYS; LYS-ARG-GLN-HIS-PRO-GLY-LYS-ARG(KRQHPGKR); LYS-ARG-GLN-HiS-PRO-GLY(KRQHPG); HAC01-Acid; TN-Ammonia; DH31-Amine-acid; P1-Aromatic; A1N-Amine-Mercaptans; A5N-Mercaptans; other compounds, anion or cation substrates (receptors), peptides; or its corresponding antibodies which can be combined with Indole or Ammonia.

In an embodiment, a kind of peptide which can be combined with Indole or Ammonia may be a predetermined protein domain (including peptide). The predetermined protein domain may use any kinds of combination methods to catch material in the air which can be identified to reach a function of air identification. In another embodiment, the predetermined protein area may be from a protein substrate wherein the protein substrate may include a hydrophobic interaction protein, a hydrogen bonding protein, or a plant hormone binding protein and the protein substrate may further include a recombinant functional homologous of the protein substrate. In an embodiment, the output unit may be a wired transmission interface, such as Universal Serial Bus (USB), Universal Asynchronous Receiver/Transmitter (UART) or Serial Peripheral Interface (SPI).

For outdoor location, GPS positioning is the method used to calculate the dog's current location, but other embodiments of the present preferred embodiment would utilize various methods of location determination, including a system integrating GPS positioning with accelerometer-based dead-reckoning. For indoor positioning, triangulation of signals generated by WiFi or Bluetooth repeaters or transceivers 13 can be used in combination with the dog's transceiver to accurate determine in-door position of the dog. Alternatively, position data produced by dead-reckoning techniques, such as an accelerometer-based method, may be used in place of the last-known position data.

In order to determine whether a GPS position data source is available, the software communicates with a GPS receiver located in electronics module 19. If at least three GPS signals are available, the software uses the time stamp obtained from each signal to calculate a pseudorange for each satellite. Once the pseudoranges have been calculated, the algorithm geometrically triangulates 63 the terrestrial position of collar 15 and records the resulting position data as the dog's current location.

If GPS signal is not available because the collar is inside a building and cannot receive satellite signals, dead-reckoning can be done. When GPS signals are not available, the position of the pet wearing the collar may also be calculated through other means, such as a dead-reckoning system incorporating an accelerometer.

Alternatively, triangulation using various Bluetooth transceivers placed inside the building can be done. RSSI localization techniques are based on measuring signal strength from a client device to several different access points, and then combining this information with a propagation model to determine the distance between the client device and the access points. Trilateration (sometimes called multilateration) techniques can be used to calculate the estimated client device position relative to the known position of access points. Fingerprinting based position can be done and is also RSSI-based, but it simply relies on the recording of the signal strength from several access points in range and storing this information in a database along with the known coordinates of the client device in an offline phase. This information can be deterministic or probabilistic. During the online tracking phase, the current RSSI vector at an unknown location is compared to those stored in the fingerprint and the closest match is returned as the estimated user location. Such systems may provide a median accuracy of 0.6 m and tail accuracy of 1.3 m. Angle of arrival based indoor positioning can also be done where a linear array of antennas receives a signal. The phase-shift difference of the received signal arriving at antennas equally separated by a “d” distance is used to compute the angle of arrival of the signal. With the advent of MIMO WiFi interfaces, which use multiple antennas, it is possible to estimate the AoA of the multipath signals received at the antenna arrays in the access points, and apply triangulation to calculate the location of client devices. Typical computation of the AoA is done with the MUSIC algorithm. Time of flight (Tof) based can be used as well. This localization approach takes timestamps provided by the wireless interfaces to calculate the ToF of signals and then use this information to estimate the distance and relative position of one client device with respect to access points. The granularity of such time measurements is in the order of nanoseconds and systems which use this technique have reported localization errors in the order of 2 m. The time measurements taken at the wireless interfaces are based on the fact that RF waves travel close to the speed of light, which remains nearly constant in most propagation media in indoor environments. Unlike traditional ToF-based echo techniques, such as those used in RADAR systems, Wi-Fi echo techniques use regular data and acknowledgement communication frames to measure the ToF. As in the RSSI approach, the ToF is used only to estimate the distance between the client device and access points. Then a trilateration technique can be used to calculate the estimated position of the device relative to the access points.

In another embodiment, accuracy of geo-position data is increased by utilizing multiple position calculations, including triangulation based on signals from GPS satellites, cell towers, and WiFi transceivers, as well as data obtained from an accelerometer-based dead-reckoning system. Additionally, a differential “receiver autonomous integrity monitoring” (“RAIM”) method may be applied to data received from the GPS, cell tower, or WiFi transceiver signals. The RAIM method utilizes data obtained from redundant sources (i.e., signal sources above the minimum number required for triangulation) to estimate the statistical probability of inaccuracy in a device's calculated geo-position. Further, the preferred embodiment of the preferred embodiment utilizes a NIST-calibrated time stamp to calculate and compensate for geo-positioning error resulting from inaccuracies in the time stamps contained in GPS, WiFi, and cell signals used for triangulation, as well as inaccuracies in the internal clock of components of electronics module 19. The preferred embodiment of the preferred embodiment utilizes NIST-calibrated time data obtained from a remote server. One example of a provider of time data with a NIST Certificate of Calibration is Certichron, Inc. A further embodiment of the preferred embodiment would utilize a nearby base station with a known location. Geo-positioning data for the local base station would be obtained via GPS, WiFi, and cell signal triangulation methods and utilized to further calculate and compensate for inaccuracies associated with the geo-position data obtained by electronics module 19. Through one or a collection of the above strategies, accurate geographical location to within a few inches for a device may be routinely obtained.

In one implementation, a user could “draw” the boundary directly onto a map of a tract of land in a software application coupled electronically with device 12 or database 23. In this embodiment, mobile device 12 would include a touch-sensitive screen apparatus and the user can simply draw the desired potty boundary. If the application determines that the dog's current position when peeing or pooping is within the defined buffer zone, the software will initiate an electrical shock or an aural reinforcement training signal to the dog, and optionally a voice training command used by the owner or trainer. Optionally, the system can also signal the owner. Even if the dog's current location is not within the buffer zone, the application also uses predictive modeling to determine whether the pet is approaching the buffer zone, based on the velocity vectors obtained from GPS/WiFi/cell tower triangulation data or data obtained from the collar's accelerometer or other dead-reckoning system. If the velocity vector data indicates that the pet will enter the buffer zone within a time period that has been pre-specified by the owner or a remote administrator (e.g., if the pet will enter the buffer zone within 5 seconds), the application will initiate 80 an aural cue and/or voice command and signal 84 the owner.

FIG. 5 may be used to illustrate the processes discussed above with respect to a pet-training scenario 90. A dog's owner 94 desires to train his pet to a portion of the owner's property having property boundary 91. The owner 94 would initiate the software application using either mobile device 12 or PC 24. Each of these devices would have access to the database stored on cloud server 23 via a WiFi router source 96 located in the owner's house 92, but it is recognized that either device could access the Internet via a Bluetooth, cell, wired, or other such method.

The owner's device would also receive signal 18 transmitting geo-positional data from pet collar 15. Upon initiation of the software application, a satellite view of the land surrounding the dog's location is displayed on a screen, and the dog's current position will be displayed as a point on the map. Drawing coordinate data from the shape file accessed previously, the screen display will also include a representation of property boundary 91 overlaid onto a satellite map image.

The owner 94 would then proceed to create a boundary 99 for the dog. In a preferred embodiment of the invention, the owner 94 would simply “draw” the boundary directly onto the map of the property in the software application. As the owner 94 selects successive points on the screen, the application would record a series of coordinates. Once the owner 94 defined the desired boundary 99 on the map of the property, the data set consisting of the series of coordinates would be used to establish that session's boundary 99. Alternatively, the owner 94 could simply walk the desired boundary line 99 while holding the collar, allowing the application to record the series of geo-position coordinates in a similar fashion.

In this sample scenario, the owner 94 has defined session boundary 99 and buffer zone 101, consisting of a set of points a particular distance (e.g., 2 feet) away from any point on boundary line 99. In an alternative embodiment of the invention, owner 94 could pinpoint a single location 98 and define the boundary 102 as a circle of a specified radius 103 with its center at the pinpointed location 98. The owner 94 could also define a buffer zone for boundary 102 as a circle of specified radius 103 minus distance 104, with its center at the pinpointed location 98.

When the dog pees or poops, the odor sensor detects the action, and the system can determine if the dog is in the correct area and if not, a shock stimulus could be delivered to the pet via shocking prongs 47. When this is done repetitively, the dog will be trained.

While certain exemplary embodiments have been described and shown in the accompanying drawings, it is to be understood that such embodiments are merely illustrative of and not restrictive on the broad invention, and that this invention should not be limited to the specific construction and arrangement shown and described, since various other modifications may occur to those ordinarily skilled in the art.

What is claimed is:

1. A pet wearable system, comprising:
a housing;
a sensor;
a pet communication module in electrical communication with the pet;
a wireless transceiver to determine a geolocation of the pet; and
a processor coupled to the pet communication module and the wireless transceiver.

2. The system of claim 1, wherein the sensor comprises a urine sensor or a feces sensor.

3. The system of claim 1, comprising a geo-fencing module wirelessly coupled to the processor through the wireless transceiver to define boundary of the predetermined area.

4. The system of claim 1, comprising a global positioning system (GPS) to provide outside coordinates.

5. The system of claim 1, comprising location beacons inside a house to provide inside building coordinates.

6. The system of claim 1, wherein the wireless transceiver comprises a cellular network, a Personal Area Network (PAN) or a wireless local area network (WLAN).

7. The system of claim 1, comprising an EEG or EKG sensor.

8. The system of claim 1, comprising a hearing aid unit.

9. The system of claim 1, comprising a display with user selectable color.

10. The system of claim 1, comprising an odor sensor to detect pet waste, wherein the odor sensor receives indoor or outdoor coordinates to advise an owner or a training network.

11. The system of claim 1, comprising code to set a geo-fence.

12. The system of claim 11, comprising code to set a radius as a geo-fence.

13. The system of claim 1, comprising code to select a point on a geographical map displayed on a screen of a device.

14. The system of claim 1, comprising, wherein said geo-position boundary comprises said data set produced by said owner by tracing out a geographical boundary map displayed on a screen of said BLUETOOTH-enabled device operated by said owner and in electrical communication with said profile held by said remote database.

15. The system of claim 1, comprising shocking prongs positioned on the housing and electrically coupled to the pet communication module to discharge electrical energy into the pet.

16. The system of claim 1, wherein the wearable housing comprises a collar, a chest strap, or a foot strap.

17. The system of claim 1, comprising code for translating user voice communication, wherein user voice communication is provided to the pet communication module.

18. The system of claim 1, comprising a flexible display coupled to the wearable housing.

19. A pet system, comprising:
a wearable housing including:
an odor sensor;
a sound generator module adapted to transmit an annoying noise to the pet, wherein said annoying noise is outside of human hearable frequency;
a wireless transceiver to determine a geolocation of the pet;
a processor coupled to the odor sensor, the sound generator, and the wireless transceiver, the processor detecting if the pet emits a predetermined odor within a preselected area and activating the sound generator in response thereto as a negative feedback to the pet; and
a geo-fencing module wirelessly coupled to the processor through the wireless transceiver to define boundary of the predetermined area.

20. The system of claim 19, wherein the wearable housing comprises a collar, a chest strap, or a foot strap.
